(19) United States
(12) Patent Application Publication
(10) Pub. No.: US 2015/0010506 A1
(43) Pub. Date: Jan. 8, 2015

(54) THERAPEUTIC PLACENTAL COMPOSITIONS, METHODS OF MAKING AND METHODS OF USE

(71) Applicant: OSIRIS THERAPEUTICS, INC., Columbia, MD (US)

(72) Inventors: Timothy Jansen, Baltimore, MD (US); Samson Tom, Basking Ridge, NJ (US); Alla Danilkovitch, Columbia, MD (US); Dana Yoo, Falls Church, MD (US); Jaime Zerhusen, Columbia, MD (US); Gabriele Putz Todd, Elkridge, MD (US); Amy Elizabeth Johnson, Baltimore, MD (US)

(73) Assignee: OSIRIS THERAPEUTICS, INC., Columbia, MD (US)

(21) Appl. No.: 14/272,339

(22) Filed: May 7, 2014

Related U.S. Application Data
(63) Continuation-in-part of application No. 14/056,101, filed on Oct. 17, 2013.
Application No. 14/056,101 is a continuation of application No. 13/030,580, filed on Feb. 18, 2011, now abandoned.
(60) Provisional application No. 61/338,464, filed on Feb. 18, 2010.
(60) Provisional application No. 61/338,489, filed on Feb. 18, 2010.
(60) Provisional application No. 61/369,562, filed on Jul. 30, 2010.

(51) Int. Cl. A61K 35/50; A61K 38/21; A61K 38/17; A61K 38/19; A61K 38/18; A61K 38/22

(52) U.S. Cl. CPC A61K 35/50; A61K 38/1825; A61K 38/1808; A61K 38/1866; A61K 38/1841; A61K 38/1858; A61K 38/1833; A61K 38/22; A61K 38/1754; A61K 38/193; A61K 38/212
USPC 424/85.7; 424/93.7; 424/85.1; 435/374

(57) ABSTRACT

This invention provides a therapeutic placental composition comprising placental cells and other placental components derived from placental tissue. A cryopreserved placental composition is also provided. The placental compositions can be used to stimulate and promote angiogenesis, reduce inflammation, and to reduce scar formation, among others. The placental tissue can optionally be an amnion, chorion, a trophoblast-depleted chorion, umbilical cord, Wharton's jelly, placental cotyledon, and/or maternal decidua. The placental composition of the present invention is useful in treating a patient with a tissue injury (e.g., wound or burn) by applying the placental composition to the injury or in close proximity. Placental compositions may also be used to promote or increase regeneration of tissue. Similar application is useful with ligament and tendon repair and for engraftment procedures such as bone engraftment.

### RELATED APPLICATIONS

This application is a continuation in part of U.S. application Ser. No. 14/056,101 entitled “Therapeutic Products Comprising Vitalized Placental Dispersions” filed on Oct. 17, 2013 which is a continuation of U.S. application Ser. No. 13/030,580 entitled “Therapeutic Products Comprising Vitalized Placental Dispersions” filed on Feb. 18, 2011. U.S. application Ser. No. 13/030,580 claimed priority to:

U.S. Provisional Application Ser. No. 61/338,464 entitled “Selectively Immunodepleted Chorionic Membranes,” filed on Feb. 18, 2010,

U.S. Provisional Application Ser. No. 61/338,489 entitled “Selectively Immunodepleted Amniotic Membranes,” filed on Feb. 18, 2010, and

U.S. Provisional Application Ser. No. 61/369,562 entitled “Therapeutic Products Comprising Vitalized Placental Dispersions,” filed on Jul. 30, 2010. The contents of the preceding applications are hereby incorporated by reference in their entireties.

This application is being co-filed on May 7, 2014 with, and incorporates by reference in their entireties, applications entitled:

“Immunocompatible Chorionic Membrane Products,” and

“Immunocompatible Amniotic Membrane Products.”

### TECHNICAL FIELD

The present technology generally relates to placental compositions, methods of medical treatment using placental compositions, and methods of making placental compositions. The present technology relates to methods and products that facilitate or improve wound healing including, for example, compositions, cryopreserved compositions, methods for promoting one or more of angiogenesis, reduction of inflammation, reduction of scar formation, reducing protease activity, promoting cell migration, promoting tissue regeneration, and inhibition of free radical oxidation.

### BACKGROUND

The structural integrity of tissue is achieved, in part, by a dynamic interaction of the tissue with bioactive molecules, extracellular matrix, and a host of cell types. Such interactions are also pivotal during tissue aging, injury, and/or restorative and regenerative treatments. For example, burns produce local tissue damage as well as systemic consequences. Currently, treatment of burn wounds, for example, is focused on promoting healing of the wound and decreasing the risk of infection. Burn wounds continue to be a frustrating and serious problem in the clinical arena, and these wounds are often accompanied by high morbidity and mortality rates. The standard of care for burns includes, for example, the use of antiseptics and gauze wound dressings. However, for severe and large surface area burns, this treatment is not satisfactory. A conventional standard for severe burn treatment continues to be autologous living skin grafts. However, the amount of skin available for grafting is often extremely limited, and this procedure always results in donor site wounds.

Attempts to improve burn wound care have included the use of a single growth factor or cocktail of growth factors as well as biological skin substitutes. Growth factors such as epidermal growth factor (EGF), platelet derived growth factor (PDGF), basic fibroblast growth factor (bFGF), vascular endothelial growth factor (VEGF), and other singular factors have been tested in burn wound healing; however, with varying results.

The use of placental membranes for burns and other types of wounds originated more than 100 years ago (as reviewed by Kesting et al., 2008). Placental membranes contain components that are present in skin and are required for wound healing such as extracellular matrix, growth factors, and cells, including MSCs (mesenchymal stem cells), for example, that are responsible for orchestrating the process of wound healing. The effectiveness of placental membranes such as amniotic membranes for burns was recorded in a number of published reports; however, the use of placental membranes for large surface area burns is limited due to challenges in providing sufficient placental membranes to cover large areas.

Another type of wound that is difficult to treat is a tunneling wound. Tunneling wounds are characterized by “tunnels” that channel from the wound into or through the muscle or subcutaneous tissue and can have one or more tunnels with varying length or depth. Causes for tunnel wound development include infection, prolonged inflammation for chronic wounds, pressure and shear forces that are concentrated where tissue layers meet, inadequate drainage absorption due to insufficient wound packing and degradation of newly granulated tissue due to too much wound packing. Dressings in a sheet form are not suitable for tunnel wounds, since the dressing will not cover the wound surface, and if placed inside the tunnel, will exert too much pressure and degrade newly granulated tissue.

What is needed in the art is a therapeutic composition(s), method of treatment(s) and/or product(s) that provides the benefits of placental membranes yet can be applied in fluid form. Moreover, there is needed a product that provides dynamic therapy throughout more than one, optimally all, of the phases of wound repair: i.e., the inflammatory, proliferative, and remodeling phases.

### SUMMARY OF THE TECHNOLOGY

The present technology provides one or more placental compositions comprising placental cells and other placental components derived from placental tissue, e.g., a whole placenta or portion thereof. The other placental components may comprise one or more therapeutic factors, extracellular matrix components, and the like. The composition(s) can be obtained by mechanical manipulation (e.g., dissection or mincing or homogenization), enzymatic digestion or combinations thereof. A placental tissue can optionally be an amnion, chorion, a mixture of amnion and chorion, or other tissue described herein, including, for example, umbilical cord tissue and Wharton's jelly. The present technology also provides a method of making one or more placental compositions.

Further, the present technology also provides one or more methods of treating a whole or partial tissue injury or defect (e.g., wound or burn) comprising administering to a patient (human or animal) in need thereof one or more placental compositions of the present technology. The present technology in some aspects also provides one or more methods of regenerating tissue comprising administering one or more placental compositions of the present technology to a patient (human or animal) in need thereof.

Optionally, the placental compositions of the present technology comprises one or more therapeutic factors set forth, for example, in Table 1, Table 2, or Table 5, among others.

Optionally, the placental cells utilized in the practice of the present technology comprise stromal cells such as MSCs (mesenchymal stem cells).

In at least one embodiment, compositions of the present technology are produced by a parallel processing method that comprises:
i) obtaining a first placental (e.g., amniotic or chorionic) tissue;
ii) obtaining placental cells from the first placental tissue;
iii) obtaining a second placental (e.g., amniotic or chorionic) tissue;
iv) disrupting the second placental tissue to form a dispersion comprising extracellular matrix, therapeutics factors, placental cells and tissue pieces, and
v) combining the placental cells and the dispersion to form one or more placental compositions and/or products.

Optionally, the first placental tissue and the second placental tissue are autologous to each other, for example, derived from the same donor.

In another embodiment, compositions of the present technology are produced by a serial processing method wherein the second placental tissue is derived from the first placental tissue after obtaining the placental cells from the first placental tissue. For example, a first chorionic tissue may be retained after isolating a population of cells thereof, and then disrupted to form a dispersion. The dispersion may then be combined with the placental cells.

Optionally, the step of isolating the placental cells can comprise the step of contacting the first placental tissue (e.g., amnion or a chorion or a chorion lacking trophoblasts) with a digestive enzyme, such as a collagenase type II, among others. Optionally, the first placental tissue is exposed to a limited digestion with an enzyme such as collagenase type II (e.g., exposure for less than about 1 hour; alternatively about 20 minutes; or alternatively about 10 minutes).

Optionally, in some embodiments, the placental tissue (from which the placental composition of the present technology is produced) is chorionic tissue depleted of trophoblasts by treatment with a digestive enzyme such as dispase followed by physical removal of the trophoblasts.

In still another embodiment, the method of making one or more placental compositions of the present technology comprises the steps of:
(i) obtaining a placental (e.g., amniotic or chorionic) tissue;
(ii) exposing the placental tissue to collagenase;
(iii) dividing the placental tissue into a first portion and a second portion;
(iv) isolating placental cells from the first placental portion;
(v) disrupting the second placental portion to form a dispersion comprising therapeutic factors, extracellular matrix, placental cells, and tissue pieces; and
(vi) combining the placental cells and the placental dispersion to form the placental composition.

In a further embodiment, the method of making one or more placental compositions of the present technology comprises the steps of:
(i) obtaining at least one placental (e.g., amniotic or chorionic) tissue;
(ii) exposing the placental tissue to at least one collagenase for a time sufficient to release placental cells;
(iii) isolating the released placental cells from the collagenase exposed placental tissue;
(iv) disrupting the collagenase exposed placental tissue to form a dispersion having one or more therapeutic factors, extracellular matrix, placental cells, and tissue pieces; and
(v) combining the placental cells and the placental dispersion to form at least one placental composition.

In one aspect, the disrupting step comprises homogenizing the collagenase exposed placental tissue. In another aspect, the disrupting step comprises mincing the collagenase exposed placental tissue. Other forms of disruption are also envisaged in the practice and performance of the present technology.

In some aspects, the one or more methods of the present technology further comprises a step of cryopreserving the placental composition wherein, after thawing, at least 40% of the placental cells in the placental composition are viable.

In some aspects of the present technology, one or more cryopreserved placental tissue compositions are provided comprising at least one disrupted placental tissue having:
(i) one or more placental cells,
(ii) one or more therapeutic factors,
(iii) one or more extracellular matrix components; and
(iv) tissue pieces comprising (i), (ii), (iii) or combinations thereof;
   wherein after subsequent thawing of the cryopreserved placental tissue composition, greater than about 40% of the placental cells are viable and the composition is depleted in functional immunogenic cells (for example, as compared to the placental tissue composition prior to such cryopreservation and subsequent thawing).

In yet other aspects, one or more cryopreserved placental tissue compositions are provided comprising:
a. at least one disrupted placental tissue including:
   (i) one or more placental cells,
   (ii) one or more therapeutic factors,
   (iii) one or more extracellular matrix components; and
   (iv) tissue pieces comprising (i), (ii), (iii) or combinations thereof; and
b. one or more cryopreservation agents,
   wherein after subsequent thawing of the cryopreserved placental tissue composition, greater than about 40% of the placental cells are viable and the composition is depleted in functional immunogenic cells; and wherein the one or more placental cells, therapeutic factors, extracellular matrix components, or combinations thereof are present in an amount effective to provide at least one therapeutic benefit.

In a still further aspect, the present technology provides at least one process for preparing one or more placental tissue compositions comprising the steps of: (a) providing a placental tissue comprising placental cells, therapeutic factors, and extracellular matrix components; (b) disrupting at least a portion of the placental tissue to form a placental dispersion comprising placental tissue pieces, placental cells, therapeutic factors and extracellular matrix components; and (c) cryopreserving the placental dispersion to form a cryopreserved placental tissue composition, wherein after subsequent thawing of the cryopreserved placental tissue composition, greater than about 40% of the placental cells are viable and wherein the composition is depleted in functional immunogenic cells.

In yet another embodiment, the present technology provides at least one process for preparing a placental tissue composition comprising the steps of:
a. providing a placental tissue having placental cells, therapeutic factors, and extracellular matrix components;
b. digesting the placental tissue with at least one enzyme to form a suspension of placental cells and placental tissue pieces;
c. separating the placental cells and the placental tissue pieces,
d. disrupting the placental tissue pieces to form a placental dispersion;
e. combining the placental cells and the placental dispersion to form a placental tissue composition; and
f. cryopreserving the placental tissue composition,
   wherein after subsequent thawing of the cryopreserved placental tissue composition, greater than about 40% of the placental cells are viable and wherein the composition is depleted in functional immunogenic cells.

In other aspects, the present technology provides a composition made by the one or more of the processes disclosed herein.

In yet further aspects, a cryopreserved placental tissue composition is provided. The cryopreserved placental tissue composition comprises:
a. disrupted placental tissue having:
   (i) one or more placental cells,
   (ii) one or more therapeutic factors,
   (iii) one or more extracellular matrix components; and
   (iv) one or more tissue pieces comprising (i), (ii), (iii) or combinations thereof; and
b. one or more cryopreservation agents,
   wherein after cryopreservation and subsequent thawing of the placental tissue composition, greater than about 40% of the placental cells are viable and the composition is depleted in functional immunogenic cells; and wherein the one or more placental cells, therapeutic factors, extracellular matrix components or combinations thereof are provided in an amount effective to do one or more of the following:
(i) reduce the amount and/or activity of pro-inflammatory cytokines;
(ii) increase the amount and/or activity of anti-inflammatory cytokines;
(iii) reduce the amount and/or activity of reactive oxygen species;
(iv) increase the amount and/or activity of anti-oxidant agents;
(v) reduce the amount and/or activity of proteases;
(vi) increase cell proliferation;
(vii) increase angiogenesis; and/or
(viii) increase cell migration.

In additional aspects, the present technology provides one or more cryopreserved placental tissue compositions comprising:
a. a minced placental tissue dispersion including:
   i. one or more placental cells,
   ii. one or more therapeutic factors, and
   iii. one or more extracellular matrix components;
   iv. one or more tissue pieces comprising (i), (ii), (iii) or combinations thereof; and
b. at least one cryopreservation agent,
   wherein after subsequent thawing of the cryopreserved placental tissue composition, greater than about 40% of the placental cells are viable and wherein the composition is substantially depleted in functional immunogenic cells.

In various embodiments of the above aspects, the composition may be stored for an extended period of time prior to subsequent thawing. In some embodiments the extended period of time can be from about 6 months to about 36 months or more, alternatively from about 6 months to at least about 24 months or greater, alternatively from about 6 months to at least about 12 months or greater, alternatively from about 6 months to about 10 months, alternatively from about 6 months, alternatively from about 3 months to about 6 months, alternatively from about 1 month to about 3 months, including other monthly and day derivations thereof for the various time periods described herein. In these embodiments, the viability of the tissue cells is substantially maintained upon subsequent thawing. In further embodiments, the viability of the tissue cells is substantially maintained for at least 24 months when stored frozen.

In yet some further aspects, the present technology provides at least one method of treating a tunnel wound of a subject (human or animal) in need thereof comprising administering to the site of the tunnel wound one or more placental compositions as described herein.

In yet another aspect, the present technology provides at least one method of treating a wound or tissue defect of a subject (human or animal) in need thereof comprising the step of administering at least one placental composition as described herein. In certain non-limiting aspects and embodiments, the amount of the composition is effective to reduce inflammation upon administration. In other aspects, the amount of the composition is effective to increase angiogenesis upon administration. In yet further aspects, the amount of the composition is effective to provide anti-oxidant conditions upon administration.

In further aspects, the present technology provides methods of promoting angiogenesis in a whole or partial wound or tissue defect comprising the steps of administering a placental composition as described herein in an amount effective to promote angiogenesis.

In yet further aspects, the present technology provides methods of preventing or reducing formation of scars comprising administering to the site in need thereof one or more placental compositions as described herein in an amount effective to prevent or reduce formation of scars.

In yet other aspects, the present technology provides methods of improving wound healing comprising administering one or more placental compositions as described herein to a subject (human or animal) in need thereof, and wherein the placental composition is provided in an effective amount to promote increased expression of one or more therapeutic factors.

In additional aspects, the present technology provides methods of directly or indirectly stimulating tissue regeneration comprising administering one or more placental compositions as described herein to a subject (human or animal) in need thereof, and wherein the placental composition is provided in an effective amount to promote increased expression of one or more therapeutic factors.

In yet further aspects, the present technology provides a method of reducing protease activity at a site in need thereof, comprising administering to the site an amount of one or more placental compositions as described herein effective to reduce the protease activity upon administration to the site.

In still further aspects, the present technology provides at least one composition comprising: a) a thawed cryopreserved placental composition; and b) a carrier.

In another aspect, the present technology provides at least one kit for treating a whole or partial wound or tissue defect comprising: a) at least one cryopreserved placental tissue composition in or on or associated with at least one pharmaceutically acceptable container; and b) instructions for administering the placental tissue composition for treating the whole or partial wound or tissue defect.

### BRIEF DESCRIPTION OF THE DRAWINGS

FIG. 1 depicts recovery of viable cells/gram of chorionic tissue isolated by digestion.

FIG. 2 depicts quantity of viable and non-viable cells isolated by digestion.

FIG. 3 depicts cell viability, before and after a freeze-thaw cycle of a placental product.

FIG. 4 depicts the level of viable cells in a placental product made with or without a digestion step of the present technology before homogenization.

FIG. 5 depicts cell phenotype of cells in a placental composition of the present technology by staining with cell surface markers CD166 (5B), CD105 (5C), and CD45 (5D) as compared with isotype control (5A).

FIG. 6 depicts cell viability using various cryoprotectants.

FIG. 7 depicts cell viability of placental composition of the present technology after storage in different cryopreservation solutions and at different time points after freezing.

FIG. 8 depicts placental tissue weight and quantity of live cells recovered following collagenase treatment of various incubation times.

FIG. 9 depicts the recovery of viable cells isolated by digestion and homogenization of amniotic membrane.

FIG. 10 depicts expression of bFGF (10A) and VEGF (10B) in placental compositions of the present technology for 14 days in culture.

FIG. 11 depicts increase in VEGF production when a placental composition of the present technology is exposed to hypoxic conditions.

FIG. 12 depicts VEGF (12A) and bFGF (12B) content in placental compositions of the present technology before freezing and after freeze/thaw.

FIG. 13 depicts expression of IFN-2α (13A) and TGF-β3 (13B) in placental compositions of the present technology.

FIG. 14 depicts TGF-β3 in multiple lots of a placental composition of the present technology.

FIG. 15A depicts detection of BMP-2, BMP-4, BMP-7, PLAB, and PLGF and FIG. 15B depicts detection of IGF-1 in placental compositions of the present technology as derived from the chorionic membrane.

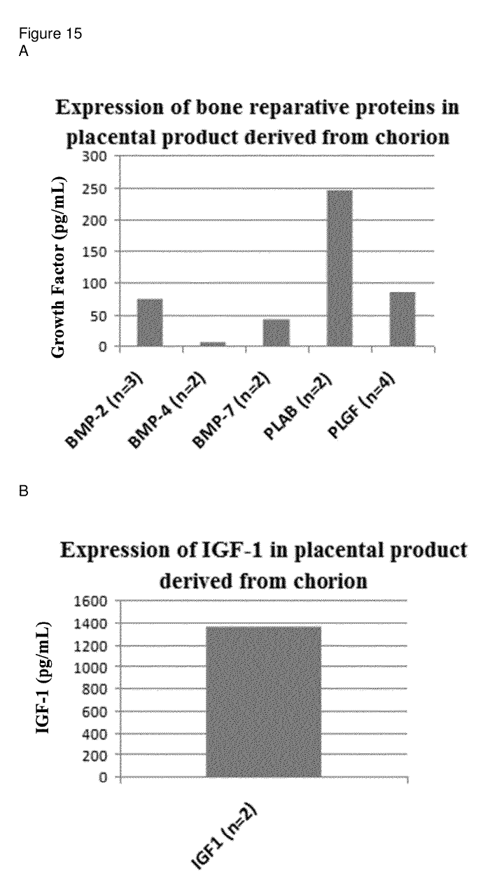

FIG. 16 depicts passage 2 cells isolated and expanded from bone marrow (16 A) or a placental composition derived from the chorionic membrane (16B) compared to cells isolated and expanded from a placental composition derived from chorionic membrane after osteoinduction (16C).

FIG. 17 depicts the quantity of viable cells (17A) and the % cell viability (17B) in multiple lots of minced placental composition of the present technology.

FIG. 18 depicts the response to an inflammatory environment by TNF-α inhibition of minced and digested placental compositions of the present technology.

FIG. 19 depicts the inhibition of elastase by minced placental compositions of the present technology.

FIG. 20 depicts the VEGF content in minced and digested placental composition of the present technology.

FIG. 21 depicts the VEGF content in minced and digested placental composition of the present technology after lysis in guanidine HCl.

FIG. 22 depicts the bFGF content in minced placental composition of the present technology after lysis in a tissue extraction buffer.

FIG. 23 depicts the sustained growth factor release of minced and digested placental composition of the present technology.

FIG. 24 depicts the proliferative capacity of placental compositions of the present technology after isolation by digestion with Serva collagenase (24A) or Worthington collagenase (24B) after 14 days in culture.

FIG. 25 depicts the compatibility of minced placental composition with a variety of osteoconductive scaffolds. FIGS. 25A and B show placental compositions on HA-TCP-Collagen Foam and FIG. 25C shows placental compositions on TransZgraft.

FIG. 26 depicts the tissue piece sizes from chorion minced with mezzaluna. Large square in upper left and right are 0.25 mm square (noted).

FIGS. 27A and B depict lipopolysaccharide (LPS) stimulated TNF-α released from various membrane preparations—Amnion+Chorion+Trophoblast (ACT), Chorion+Trophoblast (CT), Trophoblast (T), Amnion (AM), and Chorion (CM).

FIG. 28 shows expression of IL-2Rα from T-cells stimulated by choriotrophoblast (CT) which secreted high levels of TNF-α.

### DETAILED DESCRIPTION OF THE TECHNOLOGY

As used here, the following definitions and abbreviations apply.

“Chorionic tissue” or “Chorionic membrane” means the chorion or a portion thereof from placental tissue, e.g., the trophoblast, the somatic mesoderm, or combinations thereof.

“Amnionic tissue” or “Amnionic membrane” means the amnion or a portion thereof from placental tissue, e.g., the epithelium layer; the basement membrane; the compact layer; the fibroblast layer; and the intermediate (spongy) layer.

“Exemplary” (or “e.g.,” or “by example”) means a non-limiting example.

The term “placental product” and “placental composition” are used interchangeably and are the compositions described and claimed herein. “Placental composition” or “placental product” includes, but is not limited to, cells, extracellular matrix components, therapeutic factors, and tissue pieces/components containing placental cells, extracellular matrix components and/or therapeutic factors, and/or combinations thereof. The placental composition may also contain one or more cryopreservation agents. The placental product or placental compositions of the present technology can also be, for example, a graft, such as allografts or xenografts.

“Placental dispersion” means a composition or product formed by physical/mechanical disruption of placental tissue. The placental dispersion can be formed from placental tissue from which a portion of the placental cells have been isolated and removed. Alternatively, the placental dispersion can be formed from placental tissue without isolating and removing placental cells. For example, a dispersion may be in the form of a homogenate, a blend, a suspension, a colloid, or a solution, among others.

“Cellular Fraction” refers to the portion of the digested placental tissue that remains after enzymatic digestion and after the tissue fraction is removed. The cellular fraction can comprise placental cells, extracellular matrix components, therapeutic factors, fragments and combinations thereof.

“Tissue Fraction” refers to the portion of the digested placental tissue that remains after enzymatic digestion after removal from the cellular fraction. The tissue fraction can comprise tissue fragments, including, cells, extracellular matrix components, therapeutic factors and combinations thereof.

“Tissue pieces” or ‘tissue fragments” means pieces of placental tissue, and include placental cells, therapeutic factors, extracellular matrix, or combinations thereof, that are part of or imbedded in the tissue pieces.

“Placental tissue” or “placental membrane” means tissue derived from the placenta in the broadest sense of the word. Placental tissue can be a whole placenta or any portion thereof. “Portions of the placenta” is meant to include chorion, amnion, a chorion and amniotic membrane (e.g., amnio-chorion), Wharton's jelly, umbilical cord, placental cotyledons, maternal decidua and/or combinations thereof. The placental tissue may be dissected or digested (or combinations thereof) to remove portions, membrane, or structures.

“Placental cells” means any cell that can be obtained from a placenta, without regard to genetic origin (e.g., maternal vs. fetal), developmental origin (e.g., endodermal, ectodermal, or mesodermal), or differentiation. Placental cells may comprise any placental cells known in the art, for example, mesenchymal stem cells (MSCs), endometrial stromal cells (ESCs), placenta-derived mesenchymal progenitor cells, fibroblasts, epithelial cells, macrophages, and the like.

“Placental cells” are further meant to require some feature of live cells such as one or more of metabolic activity, structural integrity (e.g., exclusion of a viability stain such as trypan blue), mitotic activity, signal transduction, and the like.

“Tissue injury” means an injury of any tissue. Tissue injury can include, for example, injuries to tissues such as connective tissue (e.g., skin, cartilage, tendons, and/or ligaments, among others), bones, or other tissues or any organ. By injury, it is meant a pathology that involves or results from a mechanical insult, metabolic defect, disease or disorder, inflammation or other insult, defect, disease or disorder. Examples of such tissue injuries include, but are not limited to burns, wounds (including tunnel wounds), ulcerations, and lacerations, ablations (including laser, freezing, cryo-surgery, heat and/or electrical ablations), and/or surgical incisions, among others.

Placental compositions that are “depleted of immunogenicity,” or “depleted of immunogenic cells,” or “depleted of immunogenic factors,” or compositions that contain “depleted amounts of functional immunogenic cells” or “depleted amounts of one or more types of functional immunogenic cells” or compositions that are “depleted in amounts of functional immunogenicity” means one or more placental compositions of the present technology that retains live therapeutic cells and/or retains therapeutic efficacy for the treatment of tissue injury (or defect) yet is free, substantially free, or depleted of at least one immunogenic cell type (e.g., CD14+ macrophages, trophoblasts, and/or maternal blood cells) and/or immunogenic factor that is/are otherwise present in a native placenta, amniotic membrane or chorionic membrane. A composition (like those of the presently described technology) that is free, substantially free, or depleted of immunogenic cell types and/or immunogenic factors includes compositions that may retain some amount of immunogenic cells/factors but the retained amount is at a level that is insufficient to produce a functional response (e.g., below detectable amounts, in negligible amounts, in amounts insufficient to produce a functional immune response).

“MSC” means mesenchymal stem cells and include fetal, neonatal, adult, or post-natal. “MSCs” include amniotic MSCs (AMSCs) and chorionic MSCs (CMSCs). MSCs generally express one or more of CD73, CD70, CD90, CD105, and CD166; and generally do not express CD45 and CD34. MSCs differentiate into mesodermal lineages (osteogenic, chondrogenic, and adipogenic).

“Extracellular matrix” or “ECM” as used herein refers to any one or more components of extracellular matrix that is associated with a tissue such as, for example, placental tissues including amniotic membrane, chorionic membrane, and/or chorioamniotic membrane. The term can include structural components of the ECM, such as collagens, laminins, fibronectin, hyaluronan, dermatan sulfate, heparin sulfate, chondroitin sulfate, decorin, and elastin, as well as soluble/functional therapeutic factors that may be present in the ECM (e.g., including proteins and fragments thereof).

“Native cells” or “tissue cells” means cells that are native, resident, or endogenous to the placental membrane, i.e. cells that are not exogenously added to the placental membrane.

“Native factors” means placental membrane factors that are native, resident, or endogenous to the placental membrane, i.e. factors that are not exogenously added to the placental membrane.

“Therapeutic cells” or “beneficial cells” include cells and components present in the stromal layer, and/or the epithelial layer of the placenta and include, for example, MSCs, fibroblasts, and/or epithelial cells.

“Therapeutic factors” means placenta-, chorionic membrane-, or amniotic membrane-derived factors that promote wound healing. Therapeutic factors also encompass molecules that may be classified as cell growth factors/proteins, tissue repair factors/proteins, as well as other factors and proteins that generally promote wound healing. Non-limiting examples of therapeutic factors include antimicrobial factors, chemoattractants, remodeling proteins such as proteases and protease inhibitors, immunoregulatory factors, chemokines, cytokines, growth factors, and other factors. Therapeutic factors also include factors that promote angiogenesis, cell proliferation, and epithelialization. Non-limiting examples of such factors include TGFα, TGFβ1, TGFβ2, TGFβ3, EGF, HB-EGF, VEGF, VEGF-C, VEGF-D, HGF, PDGF-AA, PDGF-AB, PDGF-BB, PIGF, PEDF, Ang-2, IGF, IGFBP1, IGFBP2, IGFBP3, adiponectin, α2-macroglobulin, FGFs (e.g., FGF-2/bFGF, KGF, KDG/FGF-7), matrix metalloproteinases (e.g., MMP-1, MMP-2, MMP-3, MMP-7, MMP-8, MMP-9, MMP-10, MMP-13), tissue inhibitors of metalloproteinases (e.g., TIMP1, TIMP2), thrombospodins (e.g., TSP1, TSP2), fibronectin, IL-1RA, NGAL, defensin, G-CSF, LIF, IFN2α, PLAB, and SDF1b. The term “therapeutic factor” may be used interchangeably with the term “placental factor.” Exemplary therapeutic factors are listed in Table 1, Table 2, or Table 5.

“Stromal cells” refers to a mixed population of cells present (optionally in native proportions) composed of neonatal mesenchymal stem cells and neonatal fibroblasts. Both neonatal mesenchymal stem cells and neonatal fibroblasts are immunoprivileged; neither express surface proteins present on immunogenic cell types that trigger an immune response.

“Stromal layer” refers to the layers in the placental membrane that do not contain the epithelial layer.

“In vitro” describes the experiments and/or procedures performed outside of the living organism (e.g., under tissue culture conditions using artificial culture medium), including, but not limited to, culture expansion of cells.

“In vivo” describes experiments and/or procedures performed within an organism, for example, an animal or human.

A “cryopreservation agent” or “cryopreservative” or “cryoprotectant” are used interchangeably herein and are substances that help to prevent damage (e.g., cellular damage) during the freezing process. Suitable cryopreservation agents include, but are not limited to, Dimethyl Sulfoxide (DMSO), a glycerol, a glycol, a propylene glycol, an ethylene glycol, propanediol, polyethylene glycol (PEG), 1,2-propanediol (PROH) or a combination thereof. Other cryopreservation agents include, for example, one or more non-cell permeating cryopreservatives selected from, for example, polyvinyl pyrrolidione, a hydroxyethyl starch, a polysaccharide, a monosaccharide, an alginate, trehalose, raffinose, dextran, human serum albumin, ficoll, lipoproteins, polyvinyl pyrrolidone, hydroxyethyl strarch, autologous plasma or a combination thereof. Other examples of useful cryopreservatives are described in Cryopreservation (BioFiles, Volume 5, Number 4 Sigma-Aldrich® Datasheet).

A “cryopreservation solution” or “cryopreservation media” refers to a composition comprising at least one cryopreservation agent. A cryopreservation solution or media may contain further components, for example, serum albumin, pharmaceutically acceptable carriers, buffers, electrolyte solutions, or saline (e.g., phosphate buffer saline). The cryopreservation solution or media may be a solution, a slurry, suspension, etc.

The human amniotic membrane (AM) is the innermost of the fetal membranes deriving from the amniotic sac and constituting the lining of the amniotic cavity. It is approximately 0.02 to 0.5 mm thick. The AM consists of five layers: a single layer of epithelial cells rests on the basement membrane and contacts the amniotic fluid. An underlying layer of connective tissue is attached to the basement membrane. These connective tissues are comprised of three structural layers: a compact layer, a fibroblast layer (sometimes referred to as a mesenchymal layer), and a spongy layer. The spongy layer is adjacent to the cellular layer of the chorion. The amnion is essentially devoid of vasculature.

The human chorionic membrane (CM) is one of the membranes that exist during pregnancy between the developing fetus and mother. It is formed by extraembryonic mesoderm and the two layers of trophoblast and surrounds the embryo and other membranes. The chorionic villi emerge from the chorion, invade the endometrium, and allow transfer of nutrients from maternal blood to fetal blood.

### Placental Compositions or Product

Overview

It has been surprisingly discovered that one or more placental compositions of the present technology can now be produced by mechanical/physical disruption or enzymatic digestion or a combination of both to produce a medicinal product of substantial and superior therapeutic value when administered to a whole or partial tissue injury or tissue defect. The placental composition(s) of the present technology also unexpectedly exhibited several advantageous properties.

Again, in a general sense the technology described herein provides for placental compositions comprising manipulated placental tissues. For example, the placental compositions can include cryopreserved amniotic membrane compositions, cryopreserved chorionic membrane compositions, and/or cryopreserved chorioamniotic membrane compositions. In certain aspects the cryopreservation methods retain high amounts of viable placental cells (i.e., cells that are native to the placental tissue(s)) and provide for the depletion of immunogenic cells and factors associated with immunogenic cells. As such, the disclosure relates to placental compositions, and particularly compositions comprising cryopreserved disrupted amniotic, chorionic, and/or chorioamniotic membranes that comprise a combination of viable cells, therapeutic factors, extracellular matrix, and reduced immunogenicity, which find use in any number of beneficial therapeutic methods. In particular aspects discussed below, the compositions can be applied to a wound or a tissue defect, and provide amounts of viable cells, therapeutic factors, extracellular matrix that can directly or indirectly induce a change in the region to which the membrane is applied (e.g., an adaptive medicine). For example, compositions can provide for improved healing of wounds, such as chronic wounds or tunnel wounds by providing viable cells, therapeutic factors, and extracellular matrix in amounts that can provide or promote the normal stages of wound healing by any of promoting: (i) a reduction of the amount and/or activity of pro-inflammatory cytokines; (ii) an increase in the amount and/or activity of anti-inflammatory cytokines; (iii) a reduction of the amount and/or activity of reactive oxygen species; (iv) an increase in the amount and/or activity of anti-oxidant agents; (v) a reduction of the amount and/or activity of proteases; (vi) an increase in cell proliferation; (vii) an increase in angiogenesis; and/or (viii) an increase in cell migration to the wound. As a chronic wound environment can include any one or more of 1) high levels of proinflammatory cytokines, 2) low levels of anti-inflammatory cytokines, 3) high levels of proteases and low levels of their inhibitors, as well as 4) high levels of oxidants and low levels of anti-oxidant to counter balance, the characteristics and functionality of the cryopreserved compositions disclosed herein are well suited to such applications.

Fluidity.

The placental compositions of the present technology share certain properties of a fluid such as an ability to deform under an applied stress and can be quantified by measurements of viscosity. Thus, such present placental composition can be spread over a surface(s) to which it is applied. For example, one mL of placental composition can be spread topically to cover more than about 1 cm², more than about 10 cm², more than about 25 cm², more than about 50 cm², or more than about 100 cm² of skin. This fluid (i.e., flowable) property solves the problem of limited applicability of sheet-like products (e.g., skin grafts) to a variety of areas needing treatment (e.g., tunnel wounds, puncture wounds, large pressure wounds, curved surfaces, etc.). It also provides a means of rapid application. In addition to spreading, the placental composition can, for example, be injected or applied as an implant. The fluidity of the present placental compositions of the present technology now makes it practical for new uses such as application to tunnel wounds, articulating joints and curved surfaces.

Dynamic Response.

Without being bound by theory, the inventors believe that the presence of live placental cells provide to the placental composition the capacity to respond to physiologic stimuli in a manner somewhat analogous to endogenous cells in situ. Evidence of a dynamic response can include the stimulated release of therapeutic factors or changes in the therapeutic factor profile at various time points after administration.

Placental Cells

Placental cells may be obtained from any placental tissue (e.g., chorionic or amniotic). Placental cells may be obtained by processing placental tissue in any manner which retains cell viability of at least one cell type (e.g., MSCs). For example, placental cells may be isolated or purified from placental tissue (e.g., by collagenase digestion of the chorion) or may be obtained without isolation from one or more placental components (e.g., extracellular matrix).

Placental cells may be obtained by any method known in the art. Useful methods of obtaining placental cells (e.g., chorionic cells) are described, for example, by Portmann-Lanz et al. (“Placental mesenchymal stem cells as potential autologous graft for pre- and perinatal neuroregeneration”; American Journal of Obstetrics and Gynecology (2006) 194, 664-73), (“Isolation and characterization of mesenchymal cells from human fetal membranes”; Journal Of Tissue Engineering And Regenerative Medicine 2007; 1: 296-305.), and (Concise Review: Isolation and Characterization of Cells from Human Term Placenta: Outcome of the First International Workshop on Placenta Derived Stem Cells”).

In one embodiment of the present technology, placental cells are obtained by contacting placental tissue with one or more digestive enzymes, for example, by immersing placental tissue (e.g., a chorion, or placental tissue lacking trophoblasts) in a solution containing the digestive enzyme. The digestive enzyme may be any digestive enzyme known in the art. The digestive enzyme may also be a combination of enzymes. Exemplary digestive enzymes include one or more: collagenases (e.g., collagenase type I, II, III and IV), matrix metalloprotease, neutral proteases, papains, deoxyribonucleases, serine protease (e.g., trypsin, chymotrypsin, elastase), or any combination thereof).

In another embodiment of the present technology, placental cells are obtained from a chorion by contacting a chorion (e.g., a chorion lacking trophoblasts) with a collagenase (e.g., collagenase type II). The collagenase may be present in any suitable concentration, for example, about 10 U/mL to about 1000 U/mL, and in any suitable collagenase solvent, such as DMEM, and at any suitable temperature, for example 37° C. The chorion may be contacted with the digestive enzyme for any suitable period of time. Optionally, the chorion is contacted with a collagenase (e.g., collagenase type II) for less than about any of: about 16 hours, about 12 hours, about 8 hours, about 3 hrs, about 2 hr, or about 1 hr. Optionally, the chorion is contacted with the collagenase (e.g., collagenase type II) for less than about 1 hour, for example, less than about any of: about 60 min, about 50 min, about 40 min, about 30 min, about 20 min, about 15 min, about 10 min, or about 5 min. Optionally, the chorion is contacted with a collagenase for a limited period of time such that a substantial portion of the placental tissue is retained on a 100 micron filter. Optionally, after the placental cells are obtained, the chorion is disrupted to form a dispersion and the population of cells is combined with (e.g., added to) the dispersion.

In other embodiments of the present technology, the placental tissue is disrupted, for example by mincing, without contacting the placental tissue with a collagenase or similar digestive enzyme. Disrupting the placental tissue without a digestion step is less time consuming than a process that includes a digestion step and provides a minimally manipulated placental composition with a high viability of live cells after cryopreservation and subsequent thawing.

The placental compositions prepared in accordance with the present technology provide a therapeutically effective amount of viable cells without the need for ex vivo expansion of the placental cells. Although ex vivo expansion is a known method of increasing the number of viable cells in a population, such a step often leads to changes in the population make-up or distribution of cell phenotype. For example, various cells in a population may expand at different rates and expansion may also induce differentiation. Accordingly, at least one embodiment of the present technology provides a placental composition comprising placental cells derived from a placental tissue wherein the placental cells exhibits a phenotypic distribution of cells which is substantially similar to the cells of the placental tissue of origin.

Placental Dispersion

A placental dispersion may be provided by disrupting a placenta (e.g., a chorion). The disruption of placental tissue may be accomplished by any physical/mechanical method of disrupting tissue (i.e. use of a “tissue disruptor” or “means for disruption”). For example, disruption may comprise homogenization, maceration, use of a blender, crushing, or mincing, among others. Disruption may additionally or alternatively comprise shearing, dicing, or chopping. Disruption may additionally or alternatively comprise sonication.

The placental tissue may be disrupted for any suitable duration which produces a dispersion from the placenta. For example, the placenta may be disrupted (e.g., homogenized) for less than about 1 minute, about 30 seconds, about 20 sec, about 15 sec, about 10 sec, or about 5 seconds. In some aspects, for example, the placenta may be disrupted by mincing for at least about one minute, for at least about 5 minutes, for at least about 15 minutes.

In a further embodiment, the placental tissue of the present technology can be disrupted by mincing the placental tissue into approximately uniformly sized pieces to form the placental dispersion. The mincing step can be conveniently accomplished by using a tool having one or more blades effective for cutting the placental tissue into sufficiently small sized pieces to form the dispersion. For example, suitable tools for mincing the placental tissue include but are not limited to knives, scalpels, herb mincers, or a mezzaluna. In one embodiment, the minced tissue pieces are triturated (pipetted up and down through a pipette) to evenly distribute the placental pieces in the placental dispersion.

In still further aspects, the present technology provides one or more placental compositions that comprise minced placental tissue wherein the average size for the tissue pieces is from about 0.42 mm² to about 1.137 mm². In some aspects, the average size is about at least 0.42 mm². In other aspects, the average size is about 100 μm² to about 25 mm². The average sizes of the tissue pieces are able to still pass through a syringe. In some aspects, the average sizes of the tissue pieces can pass through an 18 gauge needle or larger. Other syringe gauges are also envisaged.

The placental tissue can be disrupted sufficiently to form a placental composition with fluid characteristic and yet retain viable cells. Accordingly, live cells in the placental compositions of the present technology can additionally comprise placental cells that are derived from the placental dispersion.

Therapeutic Factors

A placental composition of the present technology may comprise one or more therapeutic factors where the therapeutic factors are components of the placental dispersion or components released into the placental composition by the placental cells or a combination thereof.

It has surprisingly been discovered that the content of therapeutic factors in placental compositions made according to the present technology have an unexpected therapeutic value. Such content of therapeutic factors as taught herein is accordingly referred to here as a “therapeutic profile” and can be provided by the cells, therapeutic factors and extracellular matrix components or combinations thereof.

In at least one embodiment of the present technology, a therapeutic profile is one that provides two or more, or three or more, or four or more or greater therapeutic factors listed in Table 1, Table 2, and Table 5. Optionally, the therapeutic factors are present in an amount of about 20% to about 500% of the mean concentration set forth in Table 1, Table 2 or Table 5. Optionally, the therapeutic factors are present in an amount of about 20% to about 500% of the minimum and the maximum (respectively) of the values set forth in Table 1, Table 2 or Table 5. It should be appreciated that therapeutic factors, according to the present technology, can be placental-derived factors such as angiogenic factors, chemokines, cytokines, growth factors, matrix metalloproteases, proteases, protease inhibitor, and combinations thereof, among others. The present placental compositions can comprise any of these therapeutic factors. Thus, useful placental compositions of the present technology can have a therapeutic profile as set forth in Table 1, Table 2, or Table 5, and can have a therapeutic profile comprising for example, at least two or more therapeutic factors, at least three or more therapeutic factors, or at least four or more therapeutic factors or greater as noted above. In some embodiments, the compositions can further comprise at least one extracellular matrix component or combinations of extracellular matrix components.

The present placental compositions can optionally comprise a therapeutic profile of one or more of a PDGF (e.g., PDGF-BB), EGF, FGF, TGF-β1, TGF-β3, and VEGF and/or one or more of IL-8, IL-6, and MCP-1.

Placental compositions of the present technology can comprise a therapeutic profile of one or more therapeutic factors which promote, for example, the migration of cells into the wound area (e.g., HGF and/or KGF), optionally in combination with a growth factor such as TGF-β1. Suitable cells that migrate into the wound area include, but are not limited to, epithelial cells, endothelial cells, fibroblasts, MSC, or combinations thereof. Optionally the concentration of such therapeutic factors is about 25% of the minimum values set forth in Table 1.

In some embodiments, the placental compositions of the present technology can comprise a therapeutic profile of one or more therapeutic factors which provide anti-inflammatory cytokines that may restart, stimulate, or enhance, for example, the healing process, for example, IL-10 and PGE-2.

Placental compositions of the present technology, can comprise a therapeutic profile of therapeutic factors that are mitotic or growth promoting as well. Placental compositions of the present technology can contain HGF and KGF.

Placental compositions of the present technology can also comprise a therapeutic profile of therapeutic factors comprising one or more angiogenic factors (e.g., VEGF and/or bFGF) and can optionally additionally comprise one or more growth factors (e.g., TGF-β1 and/or TGF-β2).

Exemplary placental compositions of the present technology contain a therapeutic profile of VEGF levels greater than about 10 pg/mL or greater than about 50 pg/mL or greater than about 100 pg/mL. For example, an exemplary placental product can comprise greater than about 200 pg/mL of VEGF as detailed in Example 9. In some embodiments, the placental product of the present technology contains a therapeutic profile of VEGF levels greater than about 1000 pg/mL or greater than about 2000 pg/mL, for example, about 2000 pg/mL to about 3000 pg/mL. Exemplary placental compositions of the present technology contain a therapeutic profile of bFGF levels greater than any of about 10 or 100 or 1,000 or 10,000 pg/mL. An exemplary placental product can comprise greater than about 11,000 pg/mL of bFGF. Suitable amounts of VEGF and/or bFGF are exemplified in Example 9, 26, 27 and 28. Optionally such bFGF-comprising placental compositions of the present technology are useful for burn wound healing.

Placental compositions of the present technology can comprise a therapeutic profile of TGF-β1, TGF-β2, and/or TGF-β3. An exemplary placental composition comprises bFGF, TGF-β1, TGF-β2, and TGF-β3. Optionally, such placental compositions of the present technology are useful when the skin pathology being treated involves an inflammatory or a scaring pathology. In some exemplary embodiments, the placental product produces TGF-β1 AND TGF-β3 in ratios effective to reduce or prevent formation of scar tissue. Placental compositions of the present technology may comprise a therapeutic profile of one or more protease inhibitors, such as tissue inhibitors of matrix metalloproteinases (TIMPs), alpha-2 macroglobulin, and/or thrombospondins.

In at least one embodiment, a placental composition (e.g., derived from chorion) comprises one or more protease inhibitors.

In another one embodiment(s), a placental composition (e.g., derived from chorion) comprises one or more protease inhibitors and extracellular matrix proteins.

In at least one further embodiment, a placental composition (e.g., derived from chorion) comprises one or more protease inhibitors and viable cells.

In still further embodiment, a placental composition (e.g., derived from chorion) comprises one or more protease inhibitors, extracellular matrix proteins, and viable cells.

Without being bound by theory, the present inventors believe that the placental compositions of the present technology have enhanced efficacy compared to non-living wound healing products because the placental cells and the therapeutic factors interact with or adapt to the host environment. Such interaction or adaptation stimulates the release of therapeutic factors or changes in the placental factor profile over time after administration, resulting in a dynamic therapy that is effective in all phases of wound repair unlike conventional wound and/or non-dynamic wound therapies.

Accordingly, such placental compositions of the present technology can optionally maintain surprising integrity for extended periods of time resulting in placental compositions that require less frequent applications and superior treatment of tissue injuries (or defects) such as, for example, burns and wounds, among others. Surprisingly, the growth factors in such placental compositions can demonstrate a longer half-life in comparison to other growth factor therapies such as Amnion-derived Cellular Cytokine Solution (ACCS).

Formulation

The placental compositions of the present technology can be administered as a dermatologically acceptable pharmaceutical product. Optionally, active pharmaceutical ingredients or excipients or combinations thereof can be added with or thereto or combined with or thereto.

Viscosity.

Viscosity values that are useful and desirable according to the present technology vary as a function of the indication being treated. For example, where broad coverage (i.e. large areas of skin) or lower concentrations of placental compositions are desired, a less viscous formulation is advantageous. Examples of less viscous formulations are those of about 1,000 cP to about 50,000 cP, or about 2,000 cP to about 25,000 cP, or about 2,000 cP to about 10,000 cP, or about 5,000 cP to about 15,000 cP. Such less viscous compositions can facilitate spreading of the applied placental composition(s) of the present technology.

Where more restricted coverage or higher levels of placental compositions are desired, a more viscous formulation is advantageous. Examples of more viscous formulations are about 20,000 cP to about 200,000 cP or about 50,000 cP to about 100,000 cP.

The skilled artisan will now readily recognize that the desired viscosity can be attained according to the present technology by adjustments of the dispersion method (discussed elsewhere herein) or by selection of a carrier, such as saline or a dermatologically acceptable thickening agent and empirically determining the concentration necessary to achieve a desired viscosity or flow characteristic. The compositions can be formulated into a liquid, a solution, a gel, a slurry, or suspension, among others.

The placental compositions of the present technology can optionally include one or more antibiotics, emollients, keratolytics agents, humectants, anti-oxidants, preservatives, or combinations thereof. Other additives are also envisaged.

In at least one embodiment, a placental composition comprises albumin, such as human serum albumin (HSA) or bovine serum albumin (BSA). Optionally, the placental composition comprises an electrolyte solution, for example, to provide physiological osmolality and pH (e.g., Plasma-Lyte A). Optionally, the placental composition comprises a cryopreservation agent, such as DMSO, glycerol, glycerin, sugars, or a mixture thereof.

In another embodiment, a placental composition comprises from about 3% to about 100% by volume of at least one cryopreservation agent in the final composition, preferably about 3% to about 90%, alternatively from about 5% to about 50%, alternatively from about 5% to about 20%, alternatively from about 3% to about 10% of at least one cryopreservation agent by volume, for example, DMSO. In some embodiments, the placental composition comprises about 5% to about 20% of a cryopreservation agent and about 0% to about 15% albumin by volume. In addition, the placental composition may further comprise at least one pharmaceutically acceptable carrier, for example, saline or an electrolyte solution.

In a further embodiment, a placental composition comprises albumin, an electrolyte solution, and a cryopreservation agent. Optionally, the therapeutic composition comprises about 1% to about 15% albumin by volume and about 5% to about 20% cryopreservation agent by volume (e.g., about 10%) in the final composition. Optionally, the albumin is HSA, the electrolyte solution is Plasma-Lyte A, and the cryopreservation agent is DMSO. In some embodiments, the placental composition comprises the cryopreservation agent in an amount of about 3% to about 100% by volume of the final composition, more preferably about 3% to about 20% by volume of the final composition.

### Manufacture

Overview

A placental composition of the present technology may be manufactured from a placenta in any suitable manner that provides the technical features taught herein. Any placental tissue is useful according to the present technology. Some of the embodiments of the present technology set forth here are meant to specifically embrace placental compositions where the placental dispersion is a dispersion of chorion that is depleted of or lacking trophoblastic components. Alternatives are also envisaged.

For example, in one embodiment of the present technology, the placental dispersion and the placental cells are derived from a different placenta or different placental portion (e.g., parallel processing). It will also be appreciated that the dispersion can include a therapeutic (e.g., drug or biologic). In another, the placental dispersion and the placental cells are derived from the same placenta or the same placental portion (e.g., sequential processing).

In one embodiment, the placental composition is manufactured by the following steps comprising:
(i) obtaining a placental (e.g., chorionic or amniotic) tissue;
(ii) digesting the placental tissue with one or more matrix degrading enzymes (e.g., a collagenase, optionally collagenase type II);
(iii) obtaining placental cells from the digested placental tissue;
(iv) disrupting the digested placental tissue with a tissue disruptor to form a placental dispersion comprising therapeutic factors, extracellular matrix, placental cells, and tissue pieces; and
(v) combining the placental cells and the placental dispersion to form the placental composition.

In one aspect, the disrupting step comprises homogenizing the digested placental tissue. In other aspects, the disrupting step comprises mincing the digested placental tissue.

In another embodiment, a placental composition is manufactured by the steps comprising:
(i) obtaining a first placental (e.g., chorionic or amniotic) tissue;
(ii) digesting the first placental tissue with one or more matrix degrading enzymes (e.g., a collagenase, optionally collagenase type II);
(iii) obtaining placental cells from the digested first placental tissue;
(iv) obtaining a second placental tissue;
(v) disrupting the second placental tissue with a tissue disruptor to form a placental dispersion comprising therapeutic factors, extracellular matrix, placental cells, and tissue pieces; and
(vi) combining the placental cells and the placental dispersion to form the placental composition.

In a still further embodiment, a placental composition is manufactured by the steps comprising:
(i) obtaining a placental tissue;
(ii) mincing the tissue into approximately uniform sized pieces to form a placental dispersion that comprises placental cells, therapeutic factors and extracellular matrix components.

In some embodiments, a process for preparing a placental tissue composition according to the present technology comprises the steps of:
a. providing a placental tissue comprising placental cells, therapeutic factors, and extracellular matrix components;
b. disrupting at least a portion of the placental tissue to form a placental dispersion comprising placental tissue pieces, placental cells, therapeutic factors and extracellular matrix components; and
c. cryopreserving the placental dispersion to form a placental tissue composition, wherein after cryopreservation and subsequent thawing of the placental tissue composition, greater than 40% of the placental cells are viable and wherein the composition is depleted in functional immunogenic cells.

In yet other embodiments, a process for preparing a placental tissue composition according to the present technology comprises the steps of:
a. providing a placental tissue comprising placental cells, therapeutic factors, and extracellular matrix components;
b. digesting the placental tissue with at least one enzyme to form a suspension of placental cells and placental tissue pieces; and
c. separating the placental cells and the placental tissue pieces, and
d. disrupting the placental tissue pieces to form a dispersion; and
e. combining the placental cells and the dispersion to form a placental tissue composition; and
f. cryopreserving the placental tissue composition, wherein after cryopreservation and subsequent thawing of the placental tissue composition, greater than 40% of the placental cells are viable and wherein the composition is depleted in functional immunogenic cells.

In some aspects, after cryopreservation and subsequent thawing, at least 70% of the placental cells are viable, alternatively at least about 75%, alternatively at least about 80%, alternatively at least about 85%, alternatively at least about 90%. It should be appreciated by those skilled in the art that such a viability percentage is non-exhaustive and includes increments in between and greater than the percentages presented.

In other embodiments, the methods of the present technology further comprise the step of cryopreserving the placental compositions. The method of cryopreserving can include adding at least one cryopreservation agent to the composition, placing the composition at a temperature range of about 2° C. to about 8° C. for about 3 minutes to about 240 minutes, for example from 10 minutes to about 60 minutes; and then subsequently freezing the composition at a temperature range of about −20° C. to about −196° C. (alternatively about −45° C. to about −80° C.).

For any of the manufacturing methods, the placental tissue can be a chorion tissue such as a chorion tissue that has been processed to reduce the number of trophoblastic cells.

Additional, exemplary placental compositions of the present technology can be manufactured or provided with a bandage or wound dressing.

### Immunocompatability and Selective Depletion

In one embodiment, the placental composition is immunocompatible. Immunocompatability can be accomplished by any selective depletion step that removes immunogenic cells or factors or immunogenicity from the placenta or placental derived tissue (or amniotic membrane thereof).

In one embodiment, the placental composition is made immunocompatible by selectively depleting it of functional immunogenic cells. A placenta can be made immunocompatible by selectively reducing or removing immunogenic cells from the placenta (or amniotic membrane thereof) relative to therapeutic cells. For example, immunogenic cells can be removed by killing the immunogenic cells or by purification of the placenta therefrom.

In another embodiment, the placental composition is made immunocompatible by selectively depleting trophoblasts, for example, by removal of the trophoblast layer.

In a further embodiment, the placental composition is made immunocompatible by selective depletion of functional CD14+ macrophages, optionally as demonstrated by a substantial decrease in lipopolysaccharide (LPS) stimulation of TNFα release or by mixed lymphocyte reaction (MLR) assay.

In a still further embodiment, the placental composition is made immunocompatible by selective depletion of maternal blood cells.

In yet a still further embodiment, the placental composition is made immunocompatible by selective depletion of functional CD14+ macrophages, trophoblasts, and/or maternal blood cells.

In an additional embodiment, the placental composition is made immunocompatible by selective depletion of trophoblasts and/or CD14+ macrophages, optionally as demonstrated by a substantial decrease in LPS stimulation of TNFα release or by MLR assay.

In some embodiments of the above aspects, the depleted amounts of functional immunogenic cells produce immunogenic factors in amounts that are below levels sufficient to produce an immune response. In some embodiments, the depleted amounts of functional immunogenic cells produce immunogenic factors in amounts below detectable limits.

Trophoblast Removal

In at least one embodiment, trophoblasts are depleted or substantially removed to produce the placental tissue from which the placental cells or the placental dispersion or both are derived. Surprisingly, such a placental composition has one or more of the following superior features (among others):
a. is substantially non-immunogenic;
b. provides remarkable healing; and
c. provides enhanced therapeutic efficacy.

Trophoblasts may be removed in any suitable manner which substantially diminishes the trophoblast content of the placental composition. In one embodiment, the trophoblasts are selectively removed. Optionally, the trophoblasts are selectively removed or otherwise removed without eliminating a substantial portion of one or more therapeutic components from the chorionic membrane (e.g., MSCs, therapeutic factors, extracellular matrix, etc). Optionally, the trophoblasts are removed before isolating a population of cells and/or disrupting the placental tissue.

One method of removing trophoblasts comprises treating the placenta (e.g., chorion or amnio-chorion) with a digestive enzyme such as dispase for about 30 to about 45 minutes and separating the trophoblasts from the placenta. Optionally, the step of separating comprises mechanical separation such as scraping. A suitable method of scraping comprises scraping with a soft instrument such as a finger.

Useful methods of removing trophoblasts from a placenta (e.g., chorion) are described by Portmann-Lanz et al. (“Placental mesenchymal stem cells as potential autologous graft for pre- and perinatal neuroregeneration”; American Journal of Obstetrics and Gynecology (2006) 194, 664-73), (“Isolation and characterization of mesenchymal cells from human fetal membranes”; Journal Of Tissue Engineering And Regenerative Medicine 2007; 1: 296-305.), and (Concise Review: Isolation and Characterization of Cells from Human Term Placenta: Outcome of the First International Workshop on Placenta Derived Stem Cells”).

In one embodiment, trophoblasts are removed while retaining the basement layer, reticular layer, and/or stromal cell layer of the amniotic membrane.

In one embodiment, trophoblasts are removed before cryopreservation.

Macrophage Removal

In one embodiment, functional macrophages are depleted or removed from the placental composition. Surprisingly, such a placental compositions has one or more of the following superior features:

is substantially non-immunogenic;
provides remarkable healing; and
provides enhanced therapeutic efficacy.

Functional macrophages can be removed in any suitable manner which substantially diminishes the macrophage content of the placental composition. Optionally, the macrophages are selectively removed or otherwise removed without eliminating a substantial portion of one or more therapeutic components from the placenta (e.g., MSCs, therapeutic factors, extracellular matrix components, etc). Optionally, a majority (e.g., substantially all) of the macrophages are removed. Macrophages, include, but are not limited to, CD14+, CD11 b+, CD18+, CD40+ and CD86+.

One method of removing immune cells such as macrophages comprises killing the immune cells by rapid freezing rates such as 60-100° C./min. Another method of removing immune cells comprises killing the immune cells by holding the cells at temperatures (e.g., about 2° C. to about 8° C., e.g., “refrigerator” temperatures) for a period of time, and then freezing the immune cells at a rate of about 1° C./min.

Although immune cells can be eliminated by rapid freezing rates, such a method can also be detrimental to therapeutic cells such as stromal cells (e.g., MSCs). The present inventors have discovered a method of whereby CD14+ macrophages can be selectively killed by placing the placenta for a period of time (e.g., for at least about 3 minutes to about 240 minutes, for example, for about 10 to about 60 mins) at a temperature above freezing (e.g., incubating at about 2° C. to about 8° C.) and then freezing the placenta (e.g., incubating at about −20° C. to about −196° C., e.g., about −80° C.±5° C.). Optionally, the step of freezing comprises freezing at a rate of less than 10° C./min (e.g., less than about 5° C./min such as at about 1° C./min).

In one embodiment, the step of refrigerating comprises incubating the composition containing at least one cryopreservation agent (e.g., DMSO) for a period of time sufficient to allow the cryopreservation agent to penetrate (e.g., equilibrate with) the placental tissues. In some embodiments, the composition further comprises albumin and optionally a pharmaceutically acceptable carrier, e.g., saline or electrolyte solution. Optionally, the step of freezing comprises reducing the temperature at a rate of about 1° C./min. Optionally, the step of freezing comprises freezing at a rate of less than 10° C./min (e.g., less than about 5° C./min such as at about 1° C./min).

In one embodiment, the step of incubating the composition containing at least one cryopreservation agent (e.g., DMSO) at a temperature of about −10° C. to about 15° C. (e.g., at about 2° C. to about 8° C.) for at least about 3 minutes to about 240 minutes, for example, about any of: 10 min, 20 min, 30 min, 40 min, 50 min, 60 min, 100 min, 120 min, 180 min, 240 min. In another embodiment the step of incubating the composition containing at least one cryopreservation agent (e.g., DMSO) comprises placing the composition at a temperature of about −10° C. to about 15° C. (e.g., at about 2° C. to about 8° C.) for about any of: 10-120 min, 20-90 min, or 30-60 min, 10-240 min. Optionally, the step of freezing comprises freezing at a rate of less than 10° C./min (e.g., less than about 5° C./min such as at about 1° C./min).

Removal of Maternal Blood Cells

In one embodiment, maternal blood cells are depleted or removed from the placental composition. Surprisingly, such a placental composition has one or more of the following superior features:

is substantially non-immunogenic;
provides remarkable healing; and
provides enhanced therapeutic efficacy.

Maternal blood cells can be removed in any suitable manner which substantially diminishes such cell content of the placental composition. Optionally, the maternal blood cells are selectively removed or otherwise removed without eliminating a substantial portion of one or more therapeutic components from the placenta (e.g., therapeutic cells (e.g., MSCs), therapeutic factors, anti-oxidant agents, anti-inflammatory agents, etc).

In one embodiment, removal of maternal blood cells comprises rinsing the amniotic membrane (e.g., with buffer such as PBS) to remove gross blood clots and any excess blood cells.

In one embodiment, removal of maternal blood cells comprises treating the amniotic membrane with an anticoagulant (e.g., citrate dextrose solution).

In one embodiment, removal of maternal blood cells comprises rinsing the amniotic membrane (e.g., with buffer such as PBS or D-PBS) to remove gross blood clots and any excess blood cells, and treating the amniotic membrane with an anticoagulant (e.g., citrate dextrose solution).

In one embodiment, the chorionic membrane is retained and removal of maternal blood cells comprises separating the chorion from the placenta by cutting around the placental skirt on the side opposite of the umbilical cord. The chorion on the umbilical side of the placenta is not removed due to the vascularization on this side.

In one embodiment, the chorionic membrane is retained and removal of maternal blood cells comprises separating the chorion from the placenta by cutting around the placental skirt on the side opposite of the umbilical cord and rinsing the amniotic membrane and chorionic membrane (e.g., with buffer such as PBS or D-PBS) to remove gross blood clots and any excess blood cells.

In one embodiment, the chorionic membrane is retained and removal of maternal blood cells comprises separating the chorion from the placenta by cutting around the placental skirt on the side opposite of the umbilical cord and treating the amniotic membrane and chorionic membrane with an anticoagulant (e.g., citrate dextrose solution).

In one embodiment, the chorionic membrane is retained and removal of maternal blood cells comprises separating the chorion from the placenta by cutting around the placental skirt on the side opposite of the umbilical cord, rinsing the chorionic membrane amniotic membrane (e.g., with buffer such as PBS) to remove gross blood clots and any excess blood cells, and treating the amniotic membrane with an anticoagulant (e.g., citrate dextrose solution).

In one embodiment, the placental composition is selectively depleted of immunogenicity as demonstrated by a reduction in LPS stimulated TNF-α release. In one embodiment, the placental composition is selectively depleted of macrophages.

In one embodiment, TNF-α is depleted by killing or removal of macrophages.

In some embodiments, the level of TNF-α is less than about 350 pg/cm², alternatively less than about 225 pg/cm², alternatively less than about 100 pg/cm² or alternatively less than about 70 pg/cm² or less.

In some embodiments, TNF-α is inhibited at least about 80%, at least about 85%, at least about 90%, at least about 95%, or at least about 100%.

Immunocompatibility can be demonstrated by any means commonly known by the skilled artisan, such demonstration can be performed by the mixed Lymphocyte reaction (MLR) and by lipopolysaccharide (LPS)-induced Tumor Necrosis Factor (TNF)-α secretion.

Preservation

A placental composition of the present technology may be used fresh or may be preserved for a period of time.

In one embodiment, the placental composition is cryopreserved. The placental composition is placed in a pharmaceutically acceptable container that can withstand cryopreservation temperatures, with at least one cryopreservation agent and cryopreserved by freezing (e.g., from about −20° C. to about −196° C., such as about −80° C.). Suitable containers for cryopreservation of the placental composition are biocompatible, non-immunogenic and able to withstand temperatures of about −196° C. to about 205° C. and include vials, pouches, bottles, and bags. The placental composition (in its container) may be frozen in a Styrofoam box to control the freezing rate. Freezing may comprise storage in a cryopreservation medium containing at least one cryopreservation agent, include, but are not limited to, for example, DMSO, glycerol, sugars, or mixtures thereof. Freezing may comprise, for example, incubating the placental composition at a temperature of about 2° C. to about 8° C., for example at about 4° C., for about 3 minutes to about 240 minutes, for example, 10 min to 60 min or 30-60 min, and then incubating at about −45° C. to about −196° C., for example, −80° C. until use. The placental composition may then be thawed for use.

The placental compositions of the present technology retain a high amount of cell viability after cryopreservation and subsequent thawing. In some aspects, after thawing, at least 40% of the placental cells are viable. In some other aspects, the placental compositions retain at least about 70% viability, alternatively at least about 75% viability, alternatively about 80% viability, alternatively about 85% viability, alternatively about 90% viability, alternatively about 100% viability.

A placental composition may be formulated to include at least one cryopreservation agent before cryopreservation. Exemplary cryopreservation agents include DMSO, glycerol, and the like. The compositions may further be formulated with additional components such as albumin (e.g., HSA or BSA), an electrolyte solution (e.g., Plasma-Lyte A), or a combination thereof. Optionally, the placental compositions comprises 0% to 15% albumin by volume and about 3% to about 100%, more suitably 3% to about 50% of at least one cryopreservation agent by volume, for example, about 1% to about 15% albumin by volume and about 5% to about 100% cryopreservation agent by volume (e.g., about 5% to about 10%).

Optionally, a placental composition can be formed by the addition of cryopreserved placental cells of the present technology to a fresh (never frozen) placental dispersion or to a frozen placental dispersion or to a lyophilized placental dispersion.

Optionally, a placental composition can be formed by the addition of fresh placental cells of the present technology to a frozen placental dispersion or to a lyophilized placental dispersion.

Cryopreserved placental compositions may be thawed before use. Suitable methods of thawing would be understood by one skilled in the relevant art. The cryopreserved placental compositions may be thawed at room temperature or at about 37° C. Suitably, the cryopreserved placental compositions are thawed at a sufficiently fast rate as to retain high viability of cells (e.g., at least 40% viable cells, more preferably at least 70% viable cells). For example, a 0.3 mL cryopreserved placental composition may be thawed in less than a minute at room temperature (about 25° C.) while a 1 mL cryopreserved placental composition may be thawed at about 3 minutes at room temperature. It would be understood by one skilled in the relevant art that increasing the volume of the placental product will increase the thaw time. Further, it would be understood that the thaw time may be reduced by thawing the composition at a higher temperature (e.g., about 37° C.).

The cryopreserved compositions have a surprisingly long shelf-life or stability to retain viable cells when frozen for extended periods of time. The cryopreserved products may be store at about −20° C. to about −196° C. (e.g., about −45° C. to about −80° C.) for two years or more with retention of high viability (at least 70% retention of viable cells) once thawed. In some aspects, the cryopreserved compositions can be stored at about −20° C. to about −196° C. (e.g., about −45° C. to about −80° C.) for at least about 3 months, at least about 6 months, at least about 9 months, at least about 12 months, at least about 15 months, at least about 24 months, at least about 36 months before thawing with a high retention of viable cells (e.g., at least 40% viable cells, alternatively at least 50% viable cells, alternatively at least about 70% viable cells, alternatively at least about 80%, about 85%, 90%, or 95% viable cells).

### Methods of Use

As discussed above, the placental compositions, and particularly the cryopreserved compositions described herein (e.g., amniotic, chorionic, and/or chorioamniotic compositions) provide an amount of viable cells, therapeutic factors, and extracellular matrix components that are effective to promote a number of beneficial therapeutic activities and effects. In the methods disclosed herein the compositions may be applied and provide amounts of therapeutic factors, viable cells, and extracellular matrix and provide an environment that can promote endogenous cells to produce any number of therapeutic factors that provide the same or similar therapeutic benefit.

The placental compositions of the present technology may be used to treat any tissue injury. A method of treatment may be provided, for example, by administering to a subject in need thereof, a placental composition of the present technology. The placental compositions of the present technology may also be used to regenerate tissue, directly or indirectly.

An administration method of the present technology is topical administration. Administering the present technology can also involve administration to an internal tissue where access is gained by a surgical procedure. Alternatively, the placental composition can be injected through a syringe or needle. Placental compositions are autologous, allogeneic, or xenogeneic.

In one embodiment, a placental composition is administered to a subject to treat a wound or tissue defect. Optionally, the wound is a laceration, scrape, thermal or chemical burn, incision, puncture, or wound caused by a projectile. Optionally, the wound is an epidermal wound, skin wound, chronic wound, acute wound, external wound, internal wounds, ocular wounds, congenital wound, ulcer, or pressure ulcer. In some aspects, the wound may be tunnel wounds. Tunnel wounds may be caused by, for example, but not limited to infection, prolonged inflammation for chronic wounds, pressure and shear forces that are concentrated where tissue layers meet, inadequate drainage absorption due to insufficient wound packing and degradation of newly granulated tissue due to too much wound packing. Such wounds may be accidental or deliberate, e.g., wounds caused during or as an adjunct to a surgical procedure. Optionally, the wound is closed surgically prior to administration.

In one embodiment, the injury is a burn, such as a first-degree burn, second-degree burn (partial thickness burns), third degree burn (full thickness burns), infection of burn wound, infection of excised and unexcised burn wound, loss of epithelium from a previously grafted or healed burn, or burn wound impetigo.

In one embodiment, the injury is an ulcer, for example, a pressure ulcer, a diabetic ulcer, venous skin ulcers, or foot or leg ulcers.

In one embodiment, a placental composition is administered by applying the placental composition directly over the skin of the subject, e.g., on the stratum corneum, on the site of the wound, so that the wound is covered. In some embodiments, the placental composition is covered with a non-adhesive dressing. Additionally or alternatively, the placental composition may be administered as an implant, e.g., as a subcutaneous implant.

In one embodiment, a placental composition is administered to the epidermis to reduce rhytids or other features of aging skin. Such treatment is also usefully combined with so-called cosmetic surgery (e.g., rhinoplasty, rhytidectomy, hair restoration, etc.).

In one embodiment, a placental composition is administered to the epidermis to accelerate healing associated with a dermal ablation procedure or a dermal abrasion procedure (e.g., including laser ablation, thermal ablation, electric ablation, deep dermal ablation, sub-dermal ablation, fractional ablation, and microdermal abrasion).

Other pathologies that may be treated with placental compositions of the present technology include traumatic wounds (e.g., civilian and military wounds), surgical scars and wounds, spinal cord injury, avascular necrosis, ablations, and ischemia.

In one embodiment, a placental composition of the present technology is used in a tissue graft procedure. Optionally, the placental composition is applied to a portion of the graft which is then attached to a biological substrate (e.g., to promote healing and/or attachment to the substrate). By way of non-limiting example, tissues such as skin, cartilage, ligament, tendon, periosteum, perichondrium, pericardium, synovium, fascia, mesenter and sinew can be used as tissue graft (e.g., any natural or synthetic grafts which are biocompatible).

In one embodiment, a placental composition is used in a tendon or ligament surgery to promote healing of a tendon or ligament. Optionally, the placental composition is applied to a portion of a tendon or ligament which is attached to a bone. The surgery can be any tendon or ligament surgery, including, e.g., knee surgery, shoulder, leg surgery, arm surgery, elbow surgery, finger surgery, hand surgery, wrist surgery, toe surgery, foot surgery, ankle surgery, and the like. For example, the placental composition can be applied to a tendon or ligament in a grafting or reconstruction procedure to promote fixation of the tendon or ligament to a bone.

Through the insight of the inventors, it has surprisingly been discovered that placental compositions of the present technology provide superior treatment (e.g., healing, healing time and/or healing strength) for wounds and defects of any tissue, cartilage and bone. In some embodiments the tissue is connective tissue or nerve tissue. In some embodiments, the placental compositions are used to heal connective tissues such as tendons and ligaments. Tendon and ligament surgeries can involve the fixation of the tendon or ligament to bone. Without being bound by theory, the present inventors believe that osteogenic and/or chondrogenic potential of MSCs in the present placental compositions promotes the healing process and healing strength of bone or cartilage. In some embodiments, the placental composition may treat any type of tissue (e.g., bone, ligament, tendon, cartilage or soft tissue) wounds and injuries or any type of defects. The present inventors believe that the present placental compositions provide an alternative or adjunctive treatment to periosteum-based therapies. For example, useful periosteum based treatments are described in Chen et al. (“Enveloping the tendon graft with periosteum to enhance tendon-bone healing in a bone tunnel: A biomechanical and histologic study in rabbits”; Arthroscopy. 2003 March; 19(3):290-6), Chen et al. (“Enveloping of periosteum on the hamstring tendon graft in anterior cruciate ligament reconstruction”; Arthroscopy. 2002 May-June; 18(5):27E), Chang et al. (“Rotator cuff repair with periosteum for enhancing tendon-bone healing: a biomechanical and histological study in rabbits”; Knee Surgery, Sports Traumatology, Arthroscopy Volume 17, Number 12, 1447-1453), each of which are incorporated by reference.

As a non-limiting example of a method of tendon or ligament surgery, the placental composition is injected into the area surrounding the tendon or ligament. Optionally, the tendon is placed into a bone tunnel before it is attached to the bone.

In one embodiment, the tendon or ligament surgery is a graft procedure, wherein the placental composition is applied to the graft. Optionally, the graft is an allograft, xenograft, or an autologous graft.

In one embodiment, the tendon or ligament surgery is repair of a torn ligament or tendon, wherein the placental composition is applied to the torn ligament or tendon.

Non-limiting examples of tendons to which a placental composition can be applied include a digitorum extensor tendon, a hamstring tendon, a bicep tendon, an Achilles Tendon, an extensor tendon, and a rotator cuff tendon.

In one embodiment, a placental composition of the present technology is used to reduce fibrosis by applying the placental composition to a wound site.

In another aspect, the disclosure provides a method of promoting tissue repair and/or tissue regeneration in a subject comprising administering to the subject a composition as disclosed herein wherein the administration provides the viable therapeutic cells, extracellular matrix, and one or more therapeutic factors in an amount effective to promote tissue repair and/or tissue regeneration. In some embodiments of this aspect, the method is used in combination with a surgical procedure selected from the group consisting of a tissue graft procedure, tendon surgery, ligament surgery, bone surgery, and spinal surgery. In some embodiments, the tissue is human tissue. In further embodiments the human tissue is cartilage, skin, ligament, tendon, or bone. In this aspect and the various embodiments, the compositions may directly or indirectly stimulates tissue regeneration.

In one embodiment, a placental composition of the present technology is used as an anti-adhesion wound barrier, wherein the placental composition is applied to a wound site, for example, to reduce fibrosis (e.g., post-operative fibrosis).

Non-limiting examples of wound sites to which the placental composition can be applied include those that are surgically induced or associated with surgery involving the spine (e.g., spinal fusions), reconstructive surgery, laminectomy, knee, shoulder, or child birth, trauma related wounds or injuries, cardiovascular procedures, angiogenesis stimulation, brain/neurological procedures, hernia repair, tendon repair, bladder repair, and ophthalmic procedures. The placental compositions may also be applied or administered to treat wounds or injuries associated with other indications, including, but not limited to, osteoarthritis, inflammatory conditions (e.g., tennis elbow), bone defects, bone repair, and connective tissue repair. For example, optionally, the wound site is associated with surgery of the spine and the stromal side of the placental composition is applied to the dura (e.g., the stromal side facing the dura). Direction for such procedures, including the selection of wound sites and/or methodologies, can be found, for example, in WO 2009/132186 and US 2010/0098743, which are hereby incorporated by reference. A placental composition of the present technology can optionally be used to reduce adhesion or fibrosis of a wound. Postoperative fibrosis is a natural consequence of all surgical wound healing. By example, postoperative peridural adhesion results in tethering, traction, and compression of the thecal sac and nerve roots, which cause a recurrence of hyperesthesia that typically manifests a few months after laminectomy surgery. Repeated surgery for removal of scar tissue is associated with poor outcome and increased risk of injury because of the difficulty of identifying neural structures that are surrounded by scar tissue. Therefore, experimental and clinical studies have primarily focused on preventing the adhesion of scar tissue to the dura mater and nerve roots. Spinal adhesions have been implicated as a major contributing factor in failure of spine surgery. Fibrotic scar tissue can cause compression and tethering of nerve roots, which can be associated with recurrent pain and physical impairment.

The placental compositions disclosed herein are useful in treating a number of wounds including: tendon repair, cartilage repair (e.g., femoral condyle, tibial plateau), ACL replacement at the tunnel/bone interface, PCL tendon repair, dental tissue augmentation, fistulas (e.g., Crohn's disease, G-tube, tracheoesophogeal), missing tissue at adhesion barriers (e.g., nasal septum repair, vaginal wall repair, abdominal wall repair, tumor resection), dermal wounds (e.g., partial thickness burns, toxic epidermal necrolysis, epidermolysis bullosa, pyoderma gangrenosum, ulcers e.g., diabetic ulcers (e.g., foot), venous leg ulcers), surgical wounds, periosteum replacement, keloids, organ lacerations, epithelial defects, and repair or replacement of a tympanic membrane.

The compositions may be used to treat ocular wounds or injuries. Ocular wounds may be the result of inflammation, injury or surgery. In another aspect, the disclosure provides a method of treating an inflammatory ocular condition in a subject comprising administering to the subject a composition as disclosed herein, wherein the administration provides the viable therapeutic cells, extracellular matrix, and one or more therapeutic factors in an amount effective to treat the inflammatory ocular condition. In embodiments of this aspect the method can comprise administration of the membrane using any technique that may be directed to promote epithelialization, reduce pain, and/or to generally reduce inflammation of eye tissue. Generally, the method may be associated with eye surgery (e.g., photorefractive keratectomy (PRK)), eye trauma (e.g., lacerations, burns, or scrapes), or an eye disease that is characterized by inflammation or the treatment of which may result in an amount of inflammation in ocular tissue. Non-limiting examples of indications that include an “inflammatory ocular condition” encompassed by the method include general repair/reconstruction of the corneal or conjunctiva surface(s) such as, for example, persistent epithelial defects; corneal ulceration; corneal transplant; descemetocele; corneal perforations; defects following excision of epithelial or subepithelial lesions or tumors (conjunctival tumors, conjunctival intraeptithelial neoplasia, subepithelial lesions, band keratopathy, scars, conjunctival folds parallel to the edges of eyelids); acute chemical burns; acute keratitis; painful bullous keratopathy; partial or complete limbal stem cell deficiency (with stem cell grafting); acute Stevens-Johnson syndrome; symbelpharon; fornix reconstruction; anophthalmia; bleb revisions; scleral thinning; and pterygium (see, e.g., Meller, D., et al., Dtsch. Arztebl. Int., (2011); 108(14):243-248, incorporated herein by reference).

The composition described may provide therapeutic amounts of tissue components (e.g., cells, therapeutic factors, extracellular matrix components and combinations thereof) that are effective to promote in vivo or in vitro:
(i) a reduction in the amount and/or activity of pro-inflammatory cytokines;
(ii) an increase in the amount and/or activity of anti-inflammatory cytokines;
(iii) a reduction in the amount and/or activity of reactive oxygen species;
(iv) an increase in the amount and/or activity of anti-oxidant agents;
(v) a reduction in the amount and/or activity of proteases;
(vi) an increase in cell proliferation;
(vii) an increase in angiogenesis; and/or
(viii) an increase in cell migration.

In vitro describes the experiments and/or procedures performed outside of the living organism (e.g., under tissue culture conditions using artificial culture medium), including, but not limited to, culture expansion of cells. In vivo describes experiments and/or procedures performed within an organism, for example, an animal or human.

The composition describe may also provide a therapeutic benefit, wherein the composition provides one or more placental cells, therapeutic factors, extracellular matrix components or combinations thereof in an amount effective to:
(i) reduce the amount and/or activity of pro-inflammatory cytokines;
(ii) increase the amount and/or activity of anti-inflammatory cytokines;
(iii) reduce the amount and/or activity of proteases;
(iv) increase angiogenesis; and/or
(vi) increase cell migration.

In some aspects, the compositions are effective to stimulate tissue regeneration. Tissue regeneration may occur directly or indirectly by the application of the compositions described herein. Tissue regeneration may be directly regenerating the tissue damaged in a wound, injury or defect. Not to be bound by theory, tissue regeneration may also include indirect regeneration of tissue by stimulating the expression of therapeutic factors then stimulate the production of new tissue. Tissue regeneration may occur at, around or in the site of the wound or tissue injury.

The compositions may also be used to promote angiogenesis in a subject in need thereof, for example, near, around, in or at the site of a wound or tissue injury. Compositions may promote the upregulation or secretion of angiogenic promoting growth factors, such as, for example, vascular endothelial growth factor (VEGF) and basic Fibroblast growth factor (bFGF).

The compositions may be used to reduce inflammation. Reduction in inflammation may be shown by the increase in the expression of one or more anti-inflammatory cytokines. Further, reduction in inflammation may be shown by the decrease in pro-inflammatory cytokines.

The compositions may also provide a reduction in the amount and/or activity of reactive oxygen species or an increase in the amount and/or activity of anti-oxidant agents.

In embodiments of the above aspects relating to wound healing methods, the compositions may either directly or may indirectly promote one or more of: (i) a reduction of the amount and/or activity of pro-inflammatory cytokines; (ii) an increase in the amount and/or activity of anti-inflammatory cytokines; (iii) a reduction of the amount and/or activity of reactive oxygen species; (iv) an increase in the amount and/or activity of anti-oxidant agents; (v) a reduction of the amount and/or activity of proteases; (vi) an increase in cell proliferation; (vii) an increase in angiogenesis; and/or (viii) an increase in cell migration to the wound.

The placental compositions disclosed herein can also be used with a carrier to form a composition, for example, a biocompatible scaffold. In some aspects, the placental compositions disclosed herein can be applied to a carrier. It would be appreciated by one skilled in the art that any suitable biocompatible scaffold, carrier or bone grafting material may be used. Suitable biocompatible scaffolds and/or suitable carriers include, but are not limited to, for example, allografts, autografts, xenografts, ceramics, bioglass, calcium sulphate, demineralized bone matrix, coral, collagen, graft composites, chondronic scaffolds, synthetic scaffolds of all types, natural/biological scaffolds of all types and the like (e.g., calcium phosphates, hydroxyapatite and tricalcium phosphate, collagen/ceramic composite, PCL, PLLA, PLGA, PEG, PGA, alginates, silk, collagen, dextran gelatin, elastin, agarose, chitosan, hyaluronan, HA-TCP-Collagen, GraftJacket®, Alloderm®, PriMatrix® and others). Types thereof include, but are not limited to, other configurations such as sponges, foams, films, sheets, gels.

Suitable carriers include but are not limited to recombinant molecules such as Bone Morphogenic Protein (e.g., Infuse ((rhBMP-2), Infuse® Bone Graft and the like.

Suitable autografts include but are not limited to bone (local bone or other), platelet rich plasma (PRP), bone marrow aspirate (BMA), adipose tissue and the like.

Suitably, the composition containing a carrier may be used for surgical repair of tissues, for example, bone repair (e.g., spinal fusions), tendon repair, cartilage repair and the like.

The present technology also provides kits for the treatment of a tissue wound or defect. The kit comprises at least one dose of the placental composition of the present technology in a pharmaceutically acceptable container. Suitable containers include vials, pouches, bags, bottles, tubes and syringes. The kit can further comprise additives, such as, for example, antibiotics, emollients, humectants, anti-oxidants, preservatives, therapeutics, or combinations thereof. The kit can further include instructions for use of the kit. Other components of the kit can include, for example, a basin, bandages, dressings, adhesives, tools, scissors, scalpels, catheters, or combinations thereof.

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

The citations provided herein are hereby incorporated by reference for the cited subject matter.

In the present specification, use of the singular includes the plural except where specifically indicated.

### EXAMPLES

### Example 1

### Obtaining Placental Tissue

A whole placenta was obtained from a registered tissue bank after informed consent. The placenta was placed, with the maternal surface (rough surface) face down, on a sterile tray. The amniotic-chorionic membrane was cut and removed from the placenta. The chorionic membrane was then separated from the amnion and washed twice in D-PBS.

The chorionic membrane was then soaked in an anticoagulant (ACD-A) solution to remove blood clots and then washed again in D-PBS.

The chorionic membrane was then digested by incubation with dispase for 30 min at 37° C. The trophoblast layer was mechanically removed by scraping with fingers and the chorion was washed again in D-PBS.

The chorionic membrane was then weighed and incubated for 24 hours in an antibiotic cocktail, and washed again in D-PBS.

### Example 2

### Obtaining a Placental Composition by Digestion and Homogenization

A chorion membrane (obtained from Example 1) was digested by incubation in 200 mL of a collagenase type II solution (300 U/mL in DMEM) for 10 min at 37° C. The remaining un-digested chorionic membrane was then removed (the tissue fraction), leaving a digestion suspension containing collagenase and placental cells (the cellular fraction).

The volume and container for digestion was determined based on the need to provide a suitable digestion environment for the tissue once placed on a shaker. The digestion was carried out on a standard plate shaker set at moderate speed in a 37° C. cell culture incubator.

The cellular fraction comprising placental cells was centrifuged at 913 rcf for 5 min to separate the digestive enzyme (collagenase type II) from the placental cells. This centrifugation step may enhance cell viability by preventing over-digestion and ensure that the enzyme is washed away before homogenizing the tissue. This centrifugation step pellets the cells without damaging them, allowing the collagenase type II to be removed as supernatant.

The cells from the cellular fraction were then re-suspended in D-PBS and centrifuged again, the supernatant poured off, and the placental cells were re-suspended in a small volume (2 mL) of cryprotectant (5% DMSO in saline). Two mL provides an adequate volume to re-suspend the cells while not over-diluting the homogenized tissue fraction once the cells from that fraction have been added.

The tissue fraction was washed twice in D-PBS to remove residual digestion enzyme and placed in a homogenization container with 1 mL cryoprotectant (5% DMSO in saline) per gram of chorionic membrane. This volume was determined to be appropriate for diluting the chorion membrane enough to produce a dispersion of ideal consistency while maintaining protein concentration at clinically significant levels. The temperature of the chorionic membrane was reduced by placing the container on ice for greater than 10 min. The chorionic membrane was then homogenized twice at high speed for 5 sec using a tissue homogenizer to obtain a chorionic homogenate.

The homogenate (from the tissue fraction) was combined with the viable isolated placental cells (from the cellular fraction) and mixed thoroughly to provide a placental composition. The placental composition may be used (e.g., for therapy) fresh or may first be preserved (e.g., cryogenically) for a period of time.

The placental composition was aliquotted into vials and incubated at 4° C. for 30-60 min. The vials were then frozen at −80° C. until use.

### Example 3

### Obtaining a Placental Composition by Digestion and Mincing

A chorion membrane (obtained from Example 1 after antibiotic treatment for 18-84 hr) was digested by incubation in 200 mL of a collagenase type II solution (300 U/mL in DMEM) for 10±2 min at 37° C. The remaining un-digested chorionic membrane was then removed (the tissue fraction), leaving a digestion suspension containing collagenase and placental cells (the cellular fraction).

The volume and container for digestion was determined based on the need to provide a suitable digestion environment for the tissue once placed on a shaker. The digestion was carried out on a standard plate shaker set at moderate speed in a 37° C. cell culture incubator.

The cellular fraction comprising placental cells was combined with chilled D-PBS and centrifuged at 913 rcf for 5 min to separate the digestive enzyme (collagenase type II) from the placental cells. This centrifugation step may enhance cell viability by preventing over-digestion and ensure that the enzyme is washed away. This centrifugation step pellets the cells without damaging them, allowing the collagenase type II to be removed as supernatant.

The cells from the cellular fraction were then re-suspended in chilled D-PBS and centrifuged again, the supernatant poured off, and the placental cells were re-suspended in 2 mL of chilled D-PBS and transferred to centrifuge tube on ice packs.

The tissue fraction was washed twice in chilled D-PBS to remove residual digestion enzyme and transferred into a chilled glass dish. The chorion membrane was cut into pieces with tissue scissors then minced into small pieces with two scalpel blades by cross-slicing.

Chilled saline (an amount in mL equal to the amount in g weighed in Example 1) was added to the minced tissue fraction and pipetted up and down to thoroughly mix.

The minced tissue fraction was pulled into a large syringe, an 18 gauge needle was attached to the syringe, and the minced tissue fraction was expulsed back into the dish. The minced tissue fraction was pulled back into the syringe, an 18 gauge needle was attached to the syringe, and the minced tissue fraction was expulsed into the tube with the cellular fraction. The minced tissue fraction and cellular fraction were mixed thoroughly to provide a placental composition.

For cryopreservation, 5% DMSO was added dropwise to the placental composition with gentle swirling, then 5% HSA was added dropwise to the placental composition with gentle swirling.

The placental composition was mixed and aliquoted into vials and incubated at 4° C. for 30-60 min. The vials were then frozen at −80° C. until use.

### Example 4

### Obtaining a Placental Composition by Mincing with a Herb Mincer

A chorion membrane (obtained from Example 1 after antibiotic treatment for 18-84 hr) was transferred to a chilled glass dish, washed to remove residual antibiotic solution, and minced with a herb mincer for 6 min to achieve small, uniformly-sized pieces.

Chilled saline was added to the minced tissue according to the following calculation using the chorion membrane (CM) weight from Example 1. The minced tissue was pipetted up and down with the saline to thoroughly mix.

[Weight of CM (g)×1.8 (mL/g)]−Volume of suspension (mL)=Volume of saline to add (mL)

The minced tissue was pulled into a large syringe, an 18 gauge needle was attached to the syringe, and the minced tissue fraction was expulsed back into the dish. The minced tissue fraction was pulled back into the syringe, an 18 gauge needle was attached to the syringe, and the minced tissue fraction was expulsed into a tube with ice packs.

For cryopreservation, 5% DMSO was added dropwise to the placental composition with gentle swirling, then 5% HSA was added dropwise to the placental composition with gentle swirling.

The placental composition was mixed and aliquotted into vials and incubated at 4° C. for 30-60 min. The vials were then frozen at −80° C. until use.

### Example 5

### Obtaining a Placental Composition by Mincing with a Mezzaluna

A chorion membrane (obtained from Example 1 after antibiotic treatment for 18-84 hr) was transferred to a chilled glass dish and minced with a mezzaluna for 6 min to achieve small, uniformly-sized pieces.

The minced tissue was pulled into a large syringe, a 16 gauge needle was attached to the syringe, and the minced tissue fraction was expulsed back into the dish. The minced tissue fraction was pulled back into the syringe, a 18 gauge needle was attached to the syringe, and the minced tissue fraction was expulsed into a tube with ice packs.

For cryopreservation, 5% DMSO was added dropwise to the placental composition with gentle swirling, then 5% HSA was added dropwise to the placental composition with gentle swirling.

The placental composition was mixed and aliquoted into vials and incubated at 4° C. for 30-60 min. The vials were then frozen at −80° C. until use.

### Example 6

### Isolation of Cells without Complete Digestion of Placental Tissue

The inventors tested whether a limited collagenase type II digestion might be performed to obtain a suspension containing live cells and yet preserve the integrity of the placental tissue (e.g., preserve therapeutic factors and remaining live cells). A brief 10 minute digestion with collagenase type II left the tissue partially intact and made further handling possible. In addition, a 10 min collagenase digestion was able to produce high numbers of viable cells.

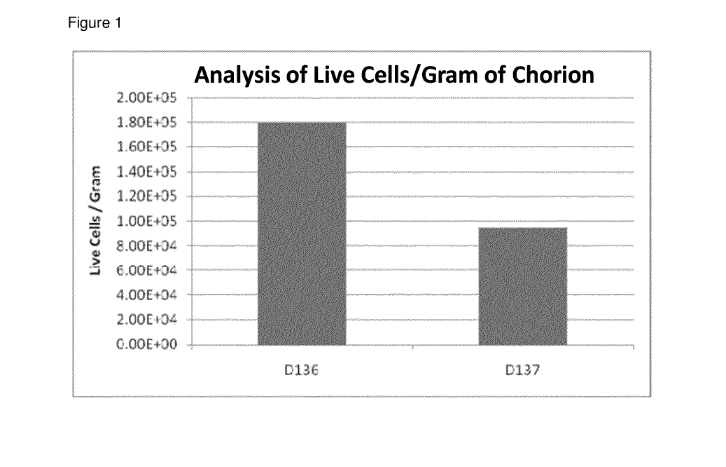

Placentas (D136, D137) were obtained and processed according to the procedure detailed in Example 1 and Example 2, except with a collagenase type II concentration of 244 U/mL, as described above. A cell count of each cellular fraction, by trypan blue staining and counting using a hemocytometer, was performed immediately following digestion to determine the number of viable cells per gram of each tissue. The data are presented in FIG. 1.

Surprisingly, a substantial population of cells was isolated by digestion of less than 1 hr (e.g., 10 min). Digesting the tissue for only 10 min allowed the loosening and removal of cells from the tissue without completely breaking up the tissue. In this manner, it was possible to separate the collagenase type II/cell mixture (cellular fraction) from the chorionic membrane without sacrificing the entire membrane by digestion. The inventors discovered that 10 min was an adequate amount of digestion time and allowed for variances introduced as a result of donor variability. The digestion process allows isolation of as many live cells as possible while not entirely disrupting the tissue integrity. The un-digested chorion membrane could then be disrupted to produce a homogenate that was rich in therapeutic factors while the cells could be isolated from the enzyme solution and then combined with the homogenate to form the placental composition.

### Example 7

### Analysis of Viable and Non-Viable Cells after Digestion

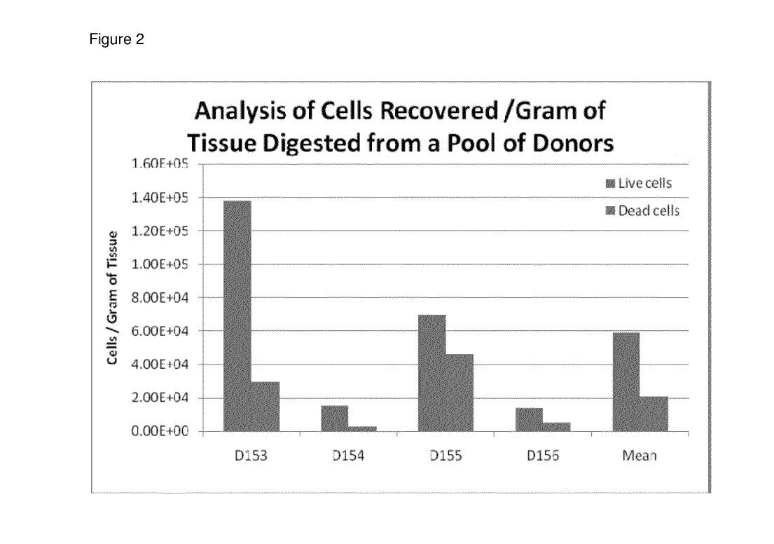

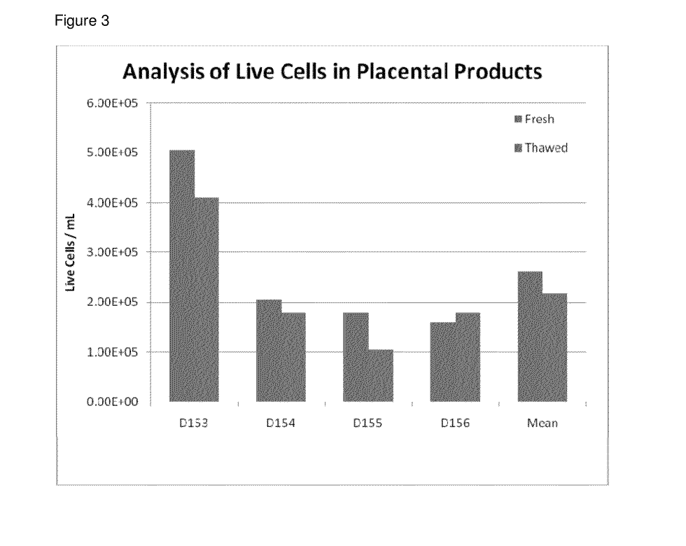

Multiple placentas were processed (D153, D154, D155, D156) and placental compositions were prepared as in Example 6. Cell counts of viable and non-viable cells were performed immediately following digestion (FIG. 2). Cell counts of viable cells were performed before freezing and after freezing and thawing (FIG. 3). Cells were counted using a hemocytometer and trypan blue staining was used to distinguish live cells. Cell count data was pooled and a mean was calculated.

As depicted in FIG. 2, digestion of an intact membrane as taught herein produces a surprising number of cells, and does so without mechanical disruption of the membrane. Also depicted in FIG. 2, digestion of a membrane as taught herein produces a surprisingly high ratio of viable to non-viable cells.

As depicted in FIG. 3, a fresh placental composition of the present technology comprises surprisingly high cell viability. Also as depicted in FIG. 3, a placental composition of the present technology subjected to a freeze-thaw cycle comprises surprisingly high cell viability. Also as depicted in FIG. 3, in a placental composition of the present technology, cell viability is retained surprisingly well after a freeze-thaw cycle.

### Example 8

### Isolation of Placental Cells with and without Digestion Before Homogenization

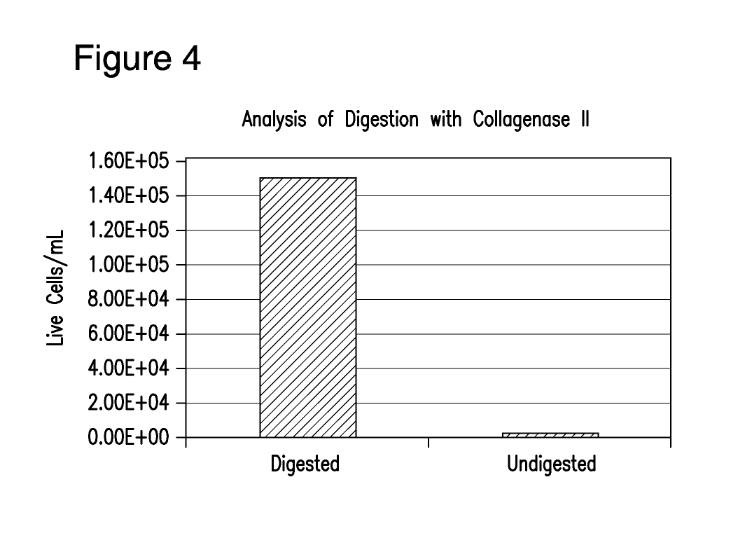

Manufacturing methods were explored to obtain superior recovery of live cells in the placental dispersion. Specifically, an experiment was performed to determine the level of viable cells in a placental composition manufactured with or without a step of cell isolation by digestion before homogenization. Briefly, a placenta was prepared according to the procedure detailed in Example 1. The resulting chorion membrane was then divided into equal halves. Half the tissue was processed as described in Example 2. The other half was processed in the same manner as the tissue fraction in Example 2 without cell isolation by collagenase type II digestion prior to the homogenization step. Cells were counted using a hemocytometer and trypan blue staining was used to distinguish live cells. The data are presented in FIG. 4.

Results indicate that cells are more easily detected in the digested tissue.

### Example 9

### Profile of a Placental Composition

Multiple placental compositions were prepared, each from a different donor, according to the procedure detailed in Example 1 and Example 2 and therapeutic factors were analyzed. Briefly, 1 mL of homogenate from each placental composition was centrifuged at 16,000 rcf in a microcentrifuge for 10 min.

The resulting supernatant from each sample was collected as a test sample. Negative control samples consisted of 5% DMSO in saline (cryopreservation solution) and positive control samples consisted of cryopreservation solution with a known concentration of spiked recombinant proteins (bFGF, EGF, and VEGF). Lists of therapeutic factors can be found in Table 3. Protein profiles comprising therapeutic factors listed in Table 1 were obtained using the SearchLight protein array assay (Aushon Biosystems). Results are indicated in Table 1 as a minimum and maximum expression levels (pg/mL) from a group of four donors. Since the supernatant is analyzed rather than the complete placental composition, it is likely that protein level estimates are below actual concentrations. The levels of VEGF and bFGF in each sample were confirmed by ELISA.

Surprisingly, many therapeutic factors were detectable at levels that are known to be influential for burn and wound healing as well as in the treatment of other indications.

As seen from the data in Table 1, placental compositions of the present technology comprise a therapeutic profile of therapeutic factors.

Only now, with the teaching herein, the skilled artisan can examine the therapeutic factors, consider the functional role as set forth in Table 2, and assess the value of a therapeutic factor in wound repair.

TABLE 1
Therapeutic Profile of Factors in the Placental Compositions of Four Donors

| | Min. | Max. | Mean | |
|---|---|---|---|---|
| Protein | (pg/mL) | (pg/mL) | (pg/mL) | Function |
| MMP1 | 2210.07 | 3468.94 | 2808.12 | Matrix and growth factor degradation, facilitate cell |
| MMP2 | 8207.46 | 70964.65 | 25648.74 | migration |
| MMP3 | 241.76 | 615.23 | 454.49 | |
| MMP7 | 79.78 | 4429.02 | 1190.31 | |
| MMP8 | 778.03 | 4661.35 | 2821.20 | |
| MMP9 | 32879.10 | 149579.10 | 71487.03 | |
| MMP10 | 6728.94 | 22686.00 | 14688.40 | |
| MMP13 | TLTD | TLTD | TLTD | |
| TIMP1 | 18739.41 | 315870.30 | 116341.69 | Inhibit activity of MMPs, angiogenic |
| TIMP2 | 7160.87 | 60711.15 | 21335.46 | |
| TSP1 | TLTD | TLTD | TLTD | Regulate TGFβ activity, anti-angiogenic |
| TSP2 | 1123.02 | 18784.67 | 6190.03 | |
| TGFα | TLTD | TLTD | TLTD | Stimulate growth and migration |
| TGFβ1 | 1041.50 | 6572.83 | 2661.65 | Promote angiogenesis, also proliferative and |
| | | | | migration stimulatory effects |
| TGFβ2 | 91.81 | 1809.81 | 558.53 | Promote angiogenesis, also proliferative and |
| | | | | migration stimulatory effects |
| TGFβ3 | 77.02 | 146.31 | 104.35 | Inhibit scar formation |
| bFGF (FGF- | 3554.58 | 11856.91 | 7479.40 | Promote angiogenesis, also proliferative and |
| 2) | | | | migration stimulatory effects |
| KGF (FGF-7) | 14.15 | 111.58 | 45.72 | Stimulate cell growth and migration |
| EGF | 0.42 | 3.72 | 1.57 | Stimulate cell growth and migration |
| HB-EGF | TLTD | TLTD | TLTD | |
| PDGFAA | 39.20 | 173.52 | 77.46 | Promote angiogenesis, also proliferative and |
| PDGFAB | 495.90 | 495.90 | 495.90 | migration stimulatory effects |
| PDGFBB | 7.73 | 235.85 | 70.56 | |
| VEGF | 13.95 | 211.17 | 76.73 | Promote angiogenesis, also proliferative and |
| VEGFC | 64.77 | 178.51 | 118.71 | migration stimulatory effects |
| VEGFD | 64.73 | 85.55 | 77.34 | |
| HGF | 9180.77 | 71280.10 | 27480.10 | Inhibit scar formation, stimulate cell growth and |
| | | | | migration |
| PEDF | 805.18 | 805.18 | 805.18 | Stimulate growth and migration |
| ANG2 | TLTD | TLTD | TLTD | Stimulate growth and migration |
| IGFBP1 | 5022.96 | 1227128.50 | 322596.69 | Regulate IGF and its proliferative effects |
| IGFBP2 | 564.62 | 564.62 | 564.62 | |
| IGFBP3 | 226.20 | 809.16 | 603.93 | |
| ACRP30 | 6403.34 | 33898.70 | 16229.15 | Regulate growth and activity of keratinocytes |
| Fibronectin | 2950999.50 | 90198200.00 | 24973399.00 | ECM, cellular adhesion, stimulates growth and |
| | | | | migration |
| Alpha2mac | 280783.30 | 4653881.00 | 1554151.49 | Inhibit protease activity, coordinate growth factor |
| | | | | bioavailability |
| IL1ra | 961.93 | 10035.52 | 3568.27 | Anti-inflammatory |
| NGAL | 420.82 | 2908.38 | 1592.17 | Anti-bacterial |
| SDF1b | TLTD | TLTD | TLTD | Recruit cells from circulation to site of tissue |
| | | | | damage |

TLTD = too low to detect

TABLE 2
Functions of Therapeutic Factors

| Specific Proteins | Selected Functions |
|---|---|
| Matrix Metalloproteinase 1 (MMP1), | Matrix and growth factor |
| MMP2, 3, 7, 8, 9, 10, 13 | degradation, facilitate cell |
| | migration |
| Tissue Inhibitors of MMPs (TIMP1 and TIMP2) | Inhibit activity of MMPs, |
| | angiogenic |
| Angiotensin-2 (Ang-2), Heparin-Bound | Stimulate growth and |
| Epidermal Growth Factor (HB-EGF), | migration |
| EGF, FGF-7 (also known as Keratinocyte | |
| Growth Factor-KGF), Placenta | |
| Growth Factor (PLGF), Pigment | |
| Epithelium Derived Factor (PEDF), | |
| Thrombopoietin (TPO), Transforming | |
| Growth Factor-α (TGF-α) | |
| Basic Fibroblast Growth Factor basic | Promote angiogenesis, also |
| (bFGF), Platelet Derived Growth | proliferative and migration |
| Factors (PDGF) AA, AB and BB, | stimulatory effects |
| Vascular Endothelial Growth Factor | |
| (VEGF), VEGF-C and VEGF-D | |
| TGF-β3, Hepatocyte Growth Factor (HGF) | Inhibit scar formation |
| α2-macroglobulin | Inhibit protease activity, |
| | coordinate growth factor |
| | bioavailability |
| Adiponectin (Acrp-30) | Regulate growth and |
| | activity of keratinocytes |
| Granulocyte Colony-Stimulating Factor (G-CSF) | Stimulate stem cell |
| | migration and proliferation |
| Interleukin 1 Receptor Antagonist (IL-1RA) | Anti-inflammatory |
| Neutrophil Gelatinase-Associated Lipocalin | Anti-bacterial |
| (N-GAL) | |
| Leukemia Inhibitory Factor (LIF) | Support of angiogenic |
| | growth factors |
| SDF-1β | Recruit cells from |
| | circulation to |
| | site of tissue damage |
| Insulin-like Growth Factor Binding | Regulate IGF and its |
| Protein (IGFBP1, 2, 3) | proliferative effects |

TABLE 3
List of selected Therapeutic Factors for Analysis.

| Function | Therapeutic Factors |
|---|---|
| Angiogenesis | Angiotensin-2 (Ang-2), Fibroblast Growth |
| | Factor basic (bFGF), heparin-bound |
| | Epidermal Growth Factor (HB-EGF), EGF, |
| | Keratinocyte Growth Factor (KGF- also |
| | known as FGF-7), Platelet derived Growth |
| | Factors (PDGF) AA, AB and BB, Vascular |
| | Endothelial Growth Factor (VEGF), VEGF-C, |
| | Hepatocyte Growth Factor (HGF), Placental |
| | Growth Factor (PLGF), Pigment Epithelium |
| | Derived Factor (PEDF), Trombospondin-1 |
| | (TSP-1), TSP-2 |
| Re-epithelialization | Epidermal Growth Factor (EGF), |
| | Keratinocyte Growth Factor (KGF), |
| | Adiponectin (Acrp-30), Insulin Growth Factor |
| | 1 (IGF), Insulin-like growth factor binding |
| | protein (IGFBP 1, 2, 3), Transforming Growth |
| | Factor α (TGFα), TGF-β1, TGF-β2 |
| Anti-microbial | Neutrophil gelatinase-associated lipocalin |
| | (NGAL), Defensin |
| Chemoattractant | Stromal Cell Derived Factor 1 Beta (SDF- |
| | 1b), bFGF, EGF, KGF |
| Extracellular Matrix | Matrix Metalloproteinase 1 (MMP1), |
| Remodeling - Proteases | MMP2, 3, 7, 8, 9, 10, 13, Tissue Inhibitors of |
| and Protease Inhibitors | MMPs (TIMP1 and 2), Alpha-2- |
| | macroglobulin, Fibronectin |
| Immunoregulatory | Granulocyte Colony-Stimulating Factor (G- |
| | CSF), Interlaukin1 receptor antagonist (IL- |
| | 1RA), Leukemia Inhibitory Factor (LIF), |
| | Interferon 2α (IFN-2α), Placental Bone |
| | Morphogenetic Protein (PLAB) |

### Example 10

### Cell Phenotype

Fluorescence Activated Cell Sorting (FACS) was performed to determine cell phenotype in multiple placental compositions of the present technology. Placental compositions were prepared according to the procedure detailed in Example 1 and Example 2. The compositions were thawed and subsequently filtered through a 100 μm filter to remove tissue debris. Single cell suspensions were then centrifuged using a Beckman TJ-6 at 2000 rpm for 10 min and washed twice with D-PBS. Supernatant was discarded after each wash, and cells were re-suspended in 2 mL of FACS staining buffer (D-PBS+0.09% NaN₃+1% FBS).

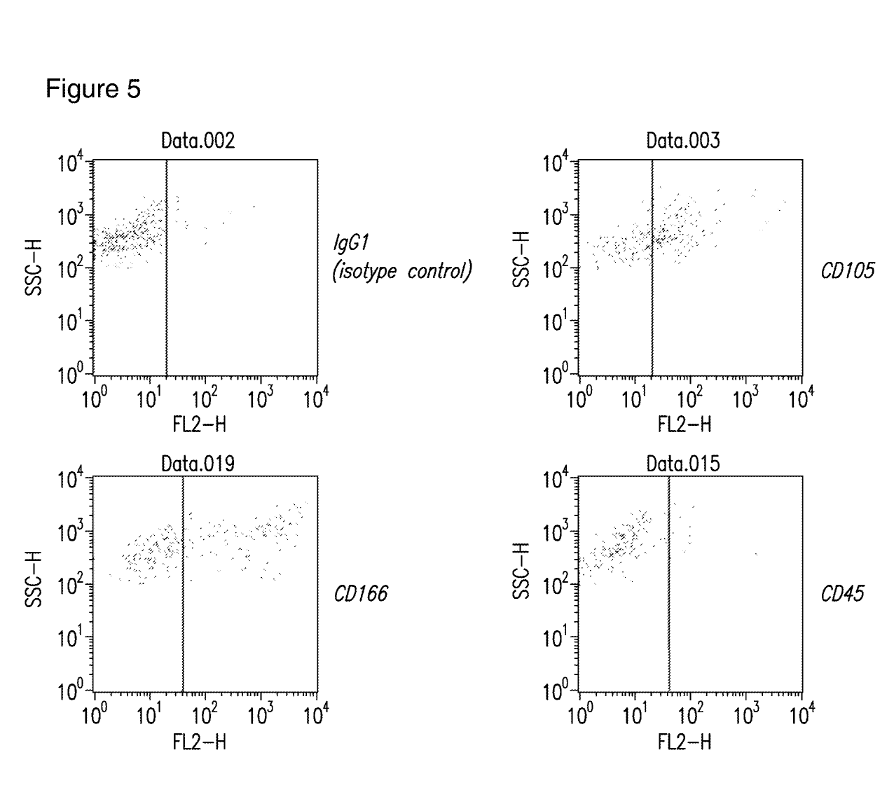

Once the single cell suspensions were prepared, a minimum of 1×10⁵ cells in 100 μL of FACS staining buffer were treated with antibodies labeled with fluorescent dye. Table 4 provides descriptions of the antibodies and the amounts used. For cell surface markers, cells were incubated for 30 min at room temperature in the dark with antibodies followed by washing twice with FACS staining buffer by centrifugation at 1300 rpm for 5 min using a Beckman TJ-6 centrifuge. Cells were then re-suspended in 400 μL of FACS staining buffer and analyzed using a BD FACSCalibur flow cytometer. Results indicate that a placental composition derived from chorion contains live cells which stain positive for MSC markers (FIG. 5), implicating the presence of MSC-like cells.

TABLE 4
FACS Antibodies

| | | Volume of | | |
|---|---|---|---|---|
| | | antibody | Cell | |
| Cell marker antibody | | solution | marker | Cell marker |
| and label type | Cat No. | used | type | specificity |
| IgG1 isotype-PE | BD 559320 | 5 μL | Cell | Isotype control |
| | | | surface | |
| CD105-PE | Caltag | 20 μL | Cell | MSC marker |
| | MHCD10504 | | surface | |
| CD166-PE | BD 559263 | 80 μL | Cell | MSC marker |
| | | | surface | |
| CD45-PE | BD 555483 | 10 μL | Cell | Hematopoetic |
| | | | surface | cell marker |

### Example 11

### Optimization of Cryoprotectants

Three placentas (D138, D139, and D140) were processed according to the procedure detailed in Example 1, and each chorionic membrane was divided in half. Each piece of chorionic tissue was processed according to the procedure detailed in Example 2 with one of the following cryoprotectants:

10% DMSO and 5% HSA in Plasma-Lyte A (CTR solution)

### 5% DMSO and 5% HSA in Plasma-Lyte A

### 5% DMSO in Saline

### 10% Glycerol in Saline

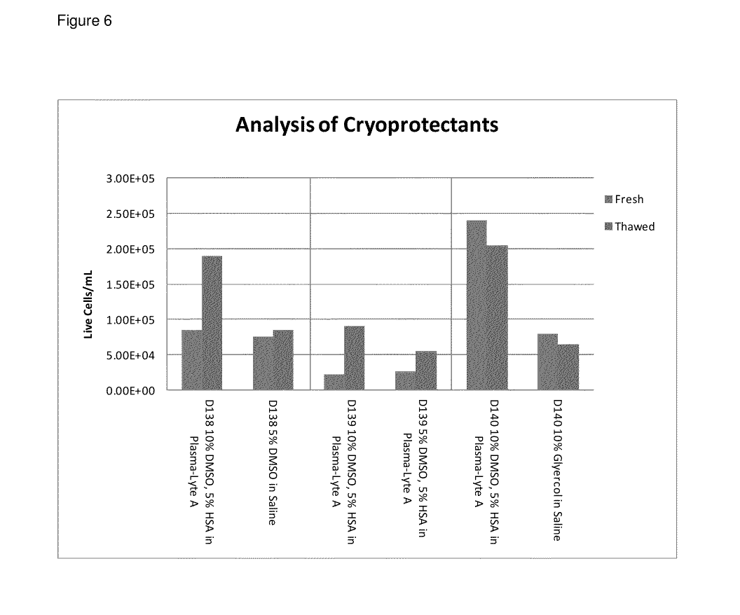

Before freezing, each fresh placental composition was counted for viable cells using a hemocytometer and trypan blue staining to distinguish live cells from dead cells. After freezing then thawing, each placental composition was counted for viable cells. The results are depicted in FIG. 6.

As depicted in FIG. 6, DMSO was a superior cryoprotectant compared to glycerol for both fresh and freeze/thawed placental composition. The use of 10% versus 5% DMSO together with 5% HSA in Plasma-Lyte A did not result in greatly larger cell numbers. 5% DMSO represents a concentration that is suitable for preserving live cell numbers after freezing and thawing and is better for patient safety.

### Example 12

### Cell Viability Three Days Up to Three Months after Freezing

Cell viability of final composition was compared using different cryoprotectant solutions and different chorionic tissue processing steps (mincing versus homogenization).

Three placentas were processed according to the procedure detailed in Example 1, and each chorionic membrane was divided into four pieces of approximately equal weight (±1 g) prior to antibiotic treatment. One quarter was used to manufacture the composition following Example 2 (Control) and frozen in 5% DMSO in saline. The remaining three pieces were processed following Example 2, except that the tissue fraction was cut into small pieces with scissors, and then minced with scalpels into a flowable consistency. Saline was added, and the samples were frozen in 5% DMSO in saline, 5% DMSO and 1% HSA in saline, or 10% DMSO and 5% HSA in saline.
Group 1=Control group (tissue fraction homogenized), 5% DMSO in saline
Group 2=Test group (tissue fraction minced), 5% DMSO in saline
Group 3=Test group (tissue fraction minced), 5% DMSO and 1% HSA in saline
Group 4=Test group (tissue fraction minced), 10% DMSO and 5% HSA in saline

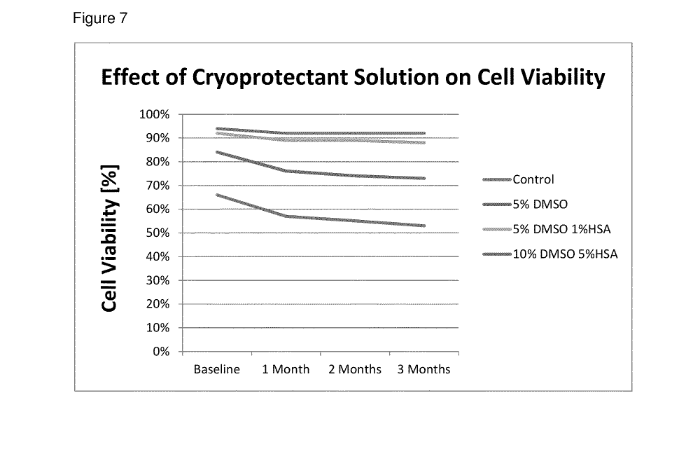

Placental composition of each group was thawed 3 days, 1 month, 2 months, and 3 months after freezing and counted for viable and dead cells using a hemocytometer and trypan blue staining. Cell viability was calculated and results of the three placentas were averaged (FIG. 7).

As depicted in FIG. 7, mincing of the tissue fraction results in higher cell viability compared to homogenization of the tissue fraction. In addition, higher cell viability was observed for placental compositions which were frozen with a cryoprotectant solution containing HSA (Group 3 and 4 with 1% to 5% HSA).

### Example 13

### Time Course Optimization of Collagenase Digestion of Chorionic Tissue

To determine the optimal time to digest a placental tissue such as chorionic tissues in collagenase type II, chorionic tissues from three different donors were analyzed. The chorions were processed according to the procedure in Example 1. Each chorionic membrane tissue was then washed twice to remove antibiotic solution and split into three pieces. Each piece of tissue was weighed to obtain an initial weight (0 min) before being digested for 10 min, 20 min, or 30 min in collagenase type II solution (300 U/mL).

At the end of each digestion period, the remaining tissue was separated from the collagenase type II solution containing the isolated cells by filtering through a 100 μm pore cell filter. The separated tissue was then weighed while the collagenase type II solution containing digested cells was centrifuged. The resulting cell pellet was re-suspended in D-PBS and counted using a hemocytometer with trypan blue exclusion.

The weight of each remaining tissue piece, including the weight of tissue remaining on the cell filter, was used to calculate the percent of weight lost by digestion with collagenase type II.

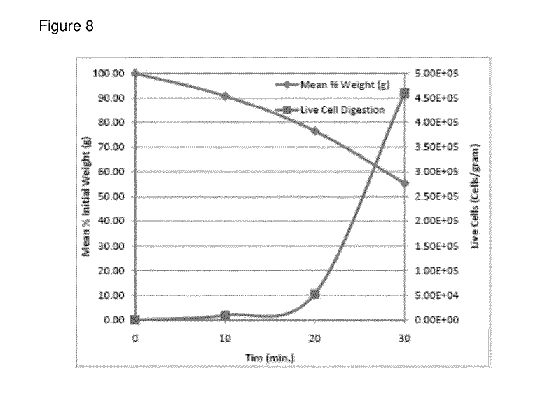

As shown in FIG. 8, after 10 min of digestion, about 10% of the original tissue weight was reduced. Further incubation resulted in a more dramatic loss of weight. By 30 minutes, nearly half of the original weight was lost. It was further noted that tissue that was digested for longer than 10 min became extremely difficult to separate from the collagenase type II solution.

FIG. 8 also shows the number of cells released by collagenase digestion. After 10 minutes of incubation, at least some of cells were released.

### Example 14

### Collagenase Digestion of Amniotic Tissue

The limited digestion method of Example 3 was tested for applicability when the placental tissue is amniotic tissue. The following procedure was performed:
1. Process placenta.
   a. Remove amniotic tissue and wash twice in D-PBS.
   b. Soak amniotic tissue to loosen red blood cells.
      i. If needed, clear red blood cells from tissue using fingers.
   c. Incubate amniotic tissue for 24 hr in antibiotic cocktail.
2. Remove amniotic tissue from antibiotic cocktail and wash twice in D-PBS.
3. Incubate amniotic tissue for 30 min at 37° C. in 200 mL trypsin solution (0.25%).
4. Remove amniotic tissue from trypsin solution and wash twice in D-PBS.
5. Incubate amniotic tissue for 10 min at 37° C. in 200 mL collagenase type II solution (300 U/mL in DMEM).
6. Remove amniotic tissue from collagenase type II solution and wash twice in D-PBS.
7. Processing of collagenase type II and trypsin live cell suspensions.
   a. Centrifuge each suspension at 913 rcf for 5 min.
   b. Pour off each supernatant and replace with 10 mL D-PBS.
      i. Re-suspend cells in D-PBS to wash.
   c. Centrifuge cell suspension at 913 rcf for 5 min.
   d. Pour off supernatants and re-suspend cells in 2 mL cryprotectant (5% DMSO in saline).
   e. Combine pellets.
8. Processing of amniotic tissue.
   a. Place amniotic tissue in homogenization container with a volume of cryoprotectant (mL) equal to the weight of the amniotic membrane (g). For example, if the amniotic membrane weighs 25 g, place it in the homogenization container with 25 mL of cryoprotectant.
   b. Allow the amniotic tissue and cryoprotectant to sit on ice for at least 10 min.
   c. Homogenize at high speed twice for 5 sec. using a tissue homogenizer.
9. Combine isolated live cells with homogenate and mix thoroughly (the “placental composition”).
10. Aliquot into vials and place at 4° C. for 30-60 min.
11. Freeze at −80° C. until use.

To determine the mean number of live cells in the amnion homogenate, multiple placentas were prepared. Each amnion was processed in one piece, and cell counts were obtained post thaw after cryopreservation (incubation at 4° C. and subsequent freezing at −80° C.). All cell count data were pooled, and a mean was calculated.

Samples from each donor were also prepared for protein array analysis. Briefly, 1 mL of homogenate from each donor was centrifuged at 16,000 rcf in a microcentrifuge for 10 min. The resulting supernatant from each sample was collected. Supernatants along with positive and negative controls were sent to Aushon Biosystems for analysis using their SearchLight protein array assay. This assay measures the levels of 37 proteins of interest in each sample. For this experiment, negative control samples consisted of 5% DMSO in saline (cryopreservation solution), and positive control samples consisted of cryopreservation solution with known concentrations of spiked recombinant proteins (bFGF, EGF, and VEGF).

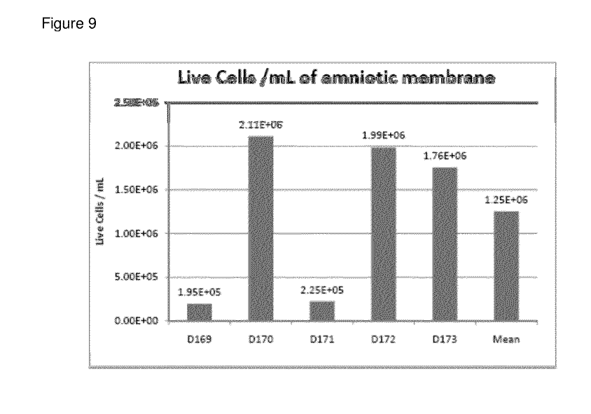

As shown in FIG. 9, limited collagenase digestion of amniotic membrane tissue resulted in release of a substantial number of live placental cells.

As shown in Table 5, limited collagenase digestion of amniotic membrane tissue preserved therapeutic factors in the placental dispersion made there from.

When Example 14 and 7 are considered together, it is now concluded that limited collagenase digestion of placental tissue, whether it be chorion tissue, amniotic tissue, or other tissue of placental origin, results unexpectedly in:

substantial numbers of release live placental cells;
preserved endogenous therapeutic factors;
preserved endogenous placental protein (e.g., matrix proteins); and
a therapeutically effective composition.

TABLE 5
Therapeutic Profiles of Amnion-Derived Placental Compositions

| | Min. | Max. | Mean |
|---|---|---|---|
| Protein | (pg/mL) | (pg/mL) | (pg/mL) |
| MMP1 | 6697.73 | 10010.27 | 8354 |
| MMP2 | 5456.52 | 53432.45 | 29444.49 |
| MMP3 | 570.97 | 579.1 | 575.04 |
| MMP7 | 74.11 | 207.31 | 140.71 |
| MMP8 | 3829.63 | 3978.42 | 3904.03 |
| MMP9 | 11735.19 | 43661.63 | 27698.41 |
| MMP10 | 38916.81 | 51526.9 | 45221.86 |
| MMP13 | TLTD | TLTD | TLTD |
| TIMP1 | 31427.94 | 78147 | 54787.47 |
| TIMP2 | 6149.25 | 23167.29 | 14658.27 |
| TSP1 | TLTD | TLTD | TLTD |
| TSP2 | 7741.98 | 13312.64 | 10527.31 |
| TGFα | TLTD | TLTD | TLTD |
| TGFβ1 | 85.17 | 350.51 | 217.84 |
| TGFβ2 | 47.98 | 58.6 | 53.29 |
| bFGF (FGF- | 19305.72 | 23427.48 | 21366.6 |
| 2) | | | |
| KGF (FGF-7) | 70.39 | 94.29 | 82.34 |
| EGF | 13.71 | 69.55 | 41.63 |
| HB-EGF | TLTD | TLTD | TLTD |
| PDGFAA | 14.47 | 27.93 | 21.2 |
| PDGFAB | TLTD | TLTD | TLTD |
| PDGFBB | 7.49 | 12.34 | 9.91 |
| VEGF | 346.3 | 1058.85 | 702.57 |
| VEGFC | 114.35 | 220.27 | 167.31 |
| VEGFD | 49.54 | 75.29 | 62.42 |
| HGF | 12068.53 | 17408.53 | 14738.53 |
| PEDF | TLTD | TLTD | TLTD |
| ANG2 | TLTD | TLTD | TLTD |
| IGFBP1 | 128.6 | 159.84 | 144.22 |
| IGFBP2 | TLTD | TLTD | TLTD |
| IGFBP3 | 699.01 | 1349.06 | 1024.04 |
| ACRP30 | 6677.35 | 11232.13 | 8954.74 |
| Fibronectin | 141595.2 | 254184.05 | 197889.63 |
| Alpha2mac | 421402.95 | 790851 | 606126.98 |
| IL1ra | 7542.74 | 10535.55 | 9039.14 |
| NGAL | 1521.63 | 3283.59 | 2402.61 |
| SDF1b | TLTD | TLTD | TLTD |

TLTD = too low to detect

### Example 15

### Live Cells from the Cellular and the Tissue Fractions of the Placental Composition

The manufacturing steps taught here (e.g., limited collagenase digestion, removal of placental cells before placental tissue disruption, and limited disruption methods) result in a highly effective therapeutic composition. The number of live cells in the final placental composition coming from the cellular fraction and the homogenized tissue fraction were evaluated.

Chorionic tissue was obtained from placental tissue of 9 subjects and the cellular fractions (e.g., collagenase-released) and homogenized tissue fractions were assessed for the number of live cells using a hemocytometer and trypan blue exclusion.

TABLE 6
Placental Cells from Cellular and Homogenized Tissue Fractions

| | | Cells in the | |
|---|---|---|---|
| | Cells in the | Homogenized Tissue | Theoretical cells in the |
| Donor | Cellular Fraction | Fraction | Placental Composition |
| D144 | 3.84E + 05 | 7.95E + 06 | 8.33E + 06 |
| D145 | 8.40E + 05 | 1.25E + 07 | 1.33E + 07 |
| D146 | 1.60E + 05 | 7.84E + 06 | 8.00E + 06 |
| D147 | 2.17E + 07 | 5.70E + 06 | 2.74E + 07 |
| D153 | 3.26E + 06 | 1.64E + 07 | 1.97E + 07 |
| D154 | 3.70E + 05 | 1.07E + 07 | 1.11E + 07 |
| D155 | 2.08E + 06 | 7.10E + 06 | 9.18E + 06 |
| D156 | 4.90E + 05 | 1.26E + 07 | 1.31E + 07 |
| Mean | 3.66E + 06 | 1.01E + 07 | 1.38E + 07 |

As shown in Table 6, 21% to 98% of the cells in the placental compositions were derived from the homogenized tissue fraction. Thus, the methods of the present invention unexpectedly preserve important therapeutic factors and live cells in the homogenized tissue fraction and also provide substantial numbers of live cells from the cellular fraction.

### Example 16

### Angiogenic Growth Factors are Expressed for a Minimum of 14 Days

Placental compositions of the present technology demonstrate a durable effect desirable for wound healing treatments. The extracellular matrix and presence of viable cells within the placental composition derived from the chorionic membrane described in this technology allow for a cocktail of proteins that are known to be important for wound healing and angiogenesis to be present for at least 14 days.

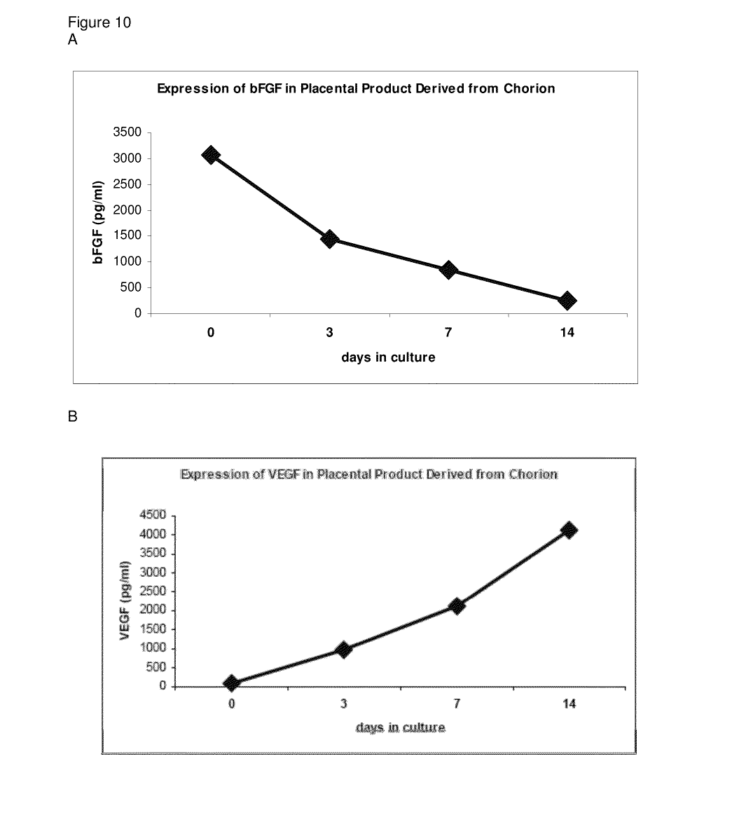

Placental compositions derived from the chorionic membrane and processed according to the procedure in Example 1 and Example 2 were thawed and plated onto tissue culture wells and incubated at 37° C.±2° C. for 3, 7, and 14 days. At each time point, a sample of the composition was collected and centrifuged at 16,000 rcf for 10 min to collect the supernatant. The supernatants were then tested by ELISA for bFGF and VEGF. FIG. 10 illustrates the duration of two key wound healing proteins, bFGF and VEGF, at 3, 7 and 14 days. Although the expression of bFGF goes down with time, it should be noted that significant levels of bFGF was present even out to 14 days. Interestingly, the expression of VEGF increased with time, which could be due to continued active expression of VEGF from the viable cells within the placental composition derived from the chorionic membrane.

### Example 17

### Response of Placental Composition to Hypoxia

Placental composition, processed according to the procedures detailed in Example 1 and Example 3, was tested for response to a hypoxic environment to mimic the hypoxic conditions found in chronic wounds.

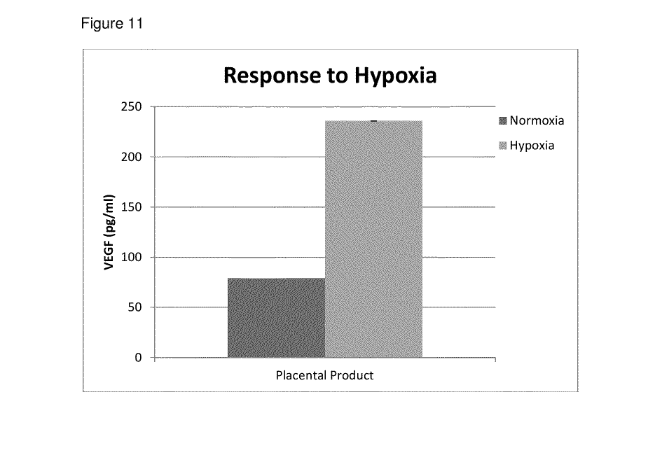

Cryopreserved placental composition was thawed and placed into the wells of two 48-well culture plates with DMEM. One plate was placed under normoxic conditions (37° C., 5% CO₂, approx. 20% O₂) and a second plate was placed under hypoxic conditions (37° C., 5% CO₂, 1% O₂). The plates were collected after 48 hours incubation under these conditions, and samples were collected. The samples were centrifuged at 16,000 rcf for 10 min in a microcentrifuge. The VEGF content of the supernatants were measured by ELISA. Results are demonstrated in FIG. 11.

As shown in FIG. 11, the placental composition responded to the hypoxic environment by increasing the production of the angiogenic growth factor VEGF by 200%.

### Example 18

### Angiogenic Growth Factor Content in Fresh v. Cryopreserved Placental Composition

The content of the angiogenic growth factors VEGF and bFGF were measured before and after cryopreservation for three lots of placental composition (D144, D145, D146) processed according to the procedures in Example 1 and Example 2.

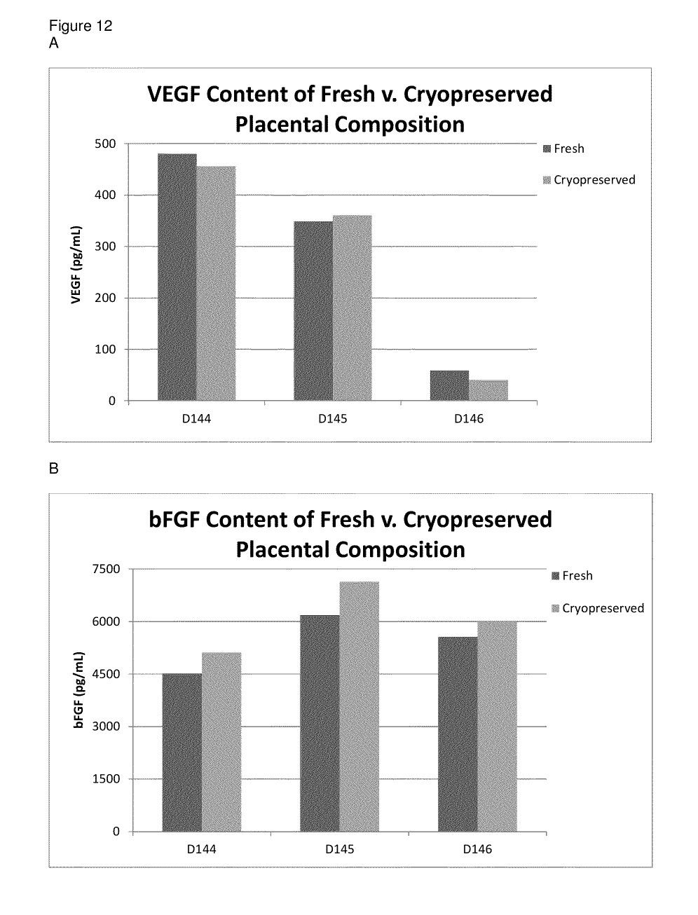

During processing, a vial of each lot of the digested placental composition was reserved before cryopreservation at −80° C. and labeled as the fresh samples. The fresh samples were centrifuged at 16,000 rcf for 10 min in a microcentrifuge. The VEGF and bFGF concentrations were measured by ELISA. From the same lots, a vial of cryopreserved placental composition was thawed after at least 12 hours stored at −80° C. (cryopreserved samples). The cryopreserved samples were treated as described for the fresh samples above. The ELISA results are shown in FIG. 12 and show that the content of both VEGF and bFGF was not altered by cryopreservation for all lots tested.

### Example 19

### Interferon-2α and Transforming Growth Factor-β3 Content in Placental Composition

Interferon-2α (IFN-2α) and Transforming Growth Factor-β3 (TGF-β3) have been described in the literature as playing critical roles in the prevention of scar and contracture formation (Kwan et al., Hand Clin, 2009, 25:511; Tredget et al., Surg Clin North Am 1997, 77:701). IFN-2α is known to decrease collagen and fibronectin synthesis and fibroblast-mediated wound contracture. Clinically, IFN-2α has been administered subcutaneously and shown to improve scar quality (Nedelec et al, Lab Clin Med 1995, 126:474). TGF-β3 regulates the deposition of extracellular matrix and has been shown to decrease scar formation when injected in rodent cutaneous wound models. Clinically, TGF-β3 has been shown to improve scar appearance when injected at the wound site (Occleston et al., J Biomater Sci Polym Ed 2008, 19:1047).

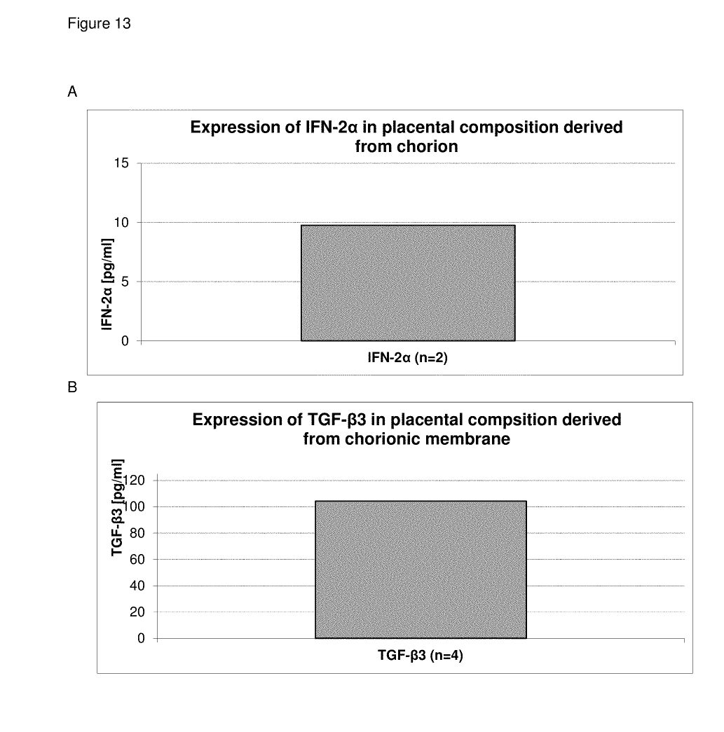

Placental compositions prepared as in Example 1 and Example 2 has been analyzed for the presence of IFN-2α and TGF-β3. Briefly, placental composition derived from the chorionic membrane was thawed and centrifuged at 16,000 rcf to collect supernatants. Supernatants were analyzed on a commercially available ELISA kit from MabTech (IFN-2α) and R&D Systems (TGF-β3). FIG. 13 shows significant expression of IFN-2α and TGF-β3 in placental compositions derived from the chorionic membrane.

### Example 20

### TGF-β3 Content in Multiple Lots of Placental Composition

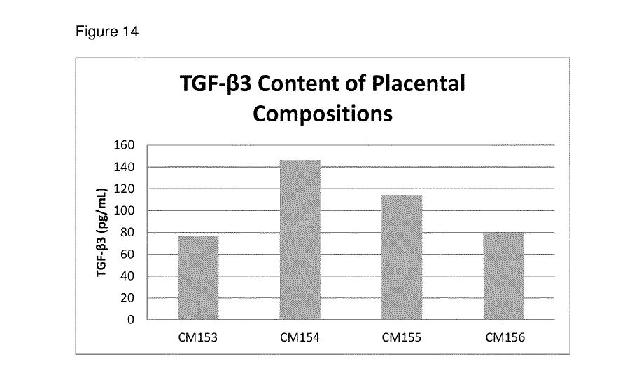

The TGF-β3 content of multiple lots of placental composition prepared as in Example 1 and Example 2 was measured. Four lots of cryopreserved placental composition were thawed and centrifuged at 16,000 rcf for 10 minutes in a microcentrifuge. The TGF-β3 concentration was measured in supernatants by ELISA. Results are shown in FIG. 14 shows expression of TGF-β3 in placental compositions.

### Example 21

### Tissue Reparative Proteins in Chorionic Placental Composition

Placental compositions derived from the chorionic membrane and processed as in Example 1 and Example 2 were analyzed for the presence of proteins that are important in tissue repair (e.g., therapeutic factors or tissue repair proteins).

Placental compositions derived from chorionic membranes described in this invention were analyzed for the presence of these tissue reparative proteins. Briefly, placental compositions derived from the chorionic membrane was incubated at 37° C.±2° C. for 72 hrs. The compositions were centrifuged, and the supernatants were analyzed on commercially available ELISA kits from R&D Systems.

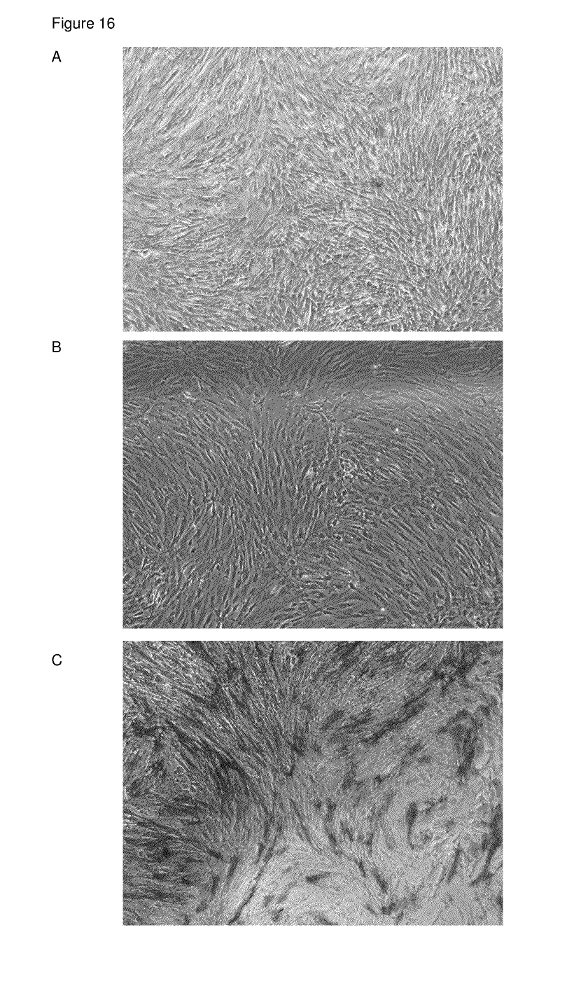

FIG. 16 shows significant expression of BMP-2, BMP-4, BMP-7, PLAB, PLGF, and IGF-1 in several donors of placental compositions derived from chorionic membranes.

Without being bound by theory, the inventors believe that efficacy of the present placental compositions for wound repair are due, in part, to the role of BMPs, IGF-1, and PLGF in the development and homeostasis of various tissues by regulating key cellular processes. BMP-2 and BMP-4 may stimulate differentiation of MSCs to osteoblasts in addition to promote cell growth; placental BMP or PLAB is a novel member of the BMP family that is suggested to mediate embryonic development. Insulin-like growth factor 1 (IGF-1) may promote proliferation and differentiation of osteoprogenitor cells. Placental derived growth factor (PLGF) may acts as a mitogen for osteoblasts.

### Example 22

### Differentiation Capacity of Cells Derived from the Chorionic Membrane

Placental cells, in optional embodiments of the present invention, are adherent, express specific cellular markers such as CD105 and lack expression of other markers such as CD45, and demonstrate the ability to differentiate into adipocytes, osteoblasts, and chondrocytes.

The expression of specific cellular markers has already been described in Example 9. To determine if the cells within the placental composition derived from the chorionic membrane can adhere to plastic and differentiate into one of the lineages, cells were isolated from the placental composition derived from the chorion as described in this invention and cultured at 37° C.±2° C. and expanded.

FIG. 16-A shows a representative image of passage 2 bone marrow MSCs, demonstrating the ability of the cells to adhere to tissue culture plastic. As a comparison, a representative image of cells isolated and expanded from human chorion membrane is shown in FIG. 16-B.

Osteogenic differentiation capacity was demonstrated by staining the cultured cells with alkaline phosphatase labeling following the manufacturer's recommendations (BCIP/NBT Alkaline Phosphatase Substrate Kit IV, Vector Laboratories Cat. No. SK-5400). Alkaline phosphatase is an enzyme involved in bone mineralization (Allori et al., Tissue Engineering: Part B, 2008, 8:275), and its expression within cells is indicative of osteo-precursor cells (Majors et al., J Orthopaedic Res, 1997, 15:546). Staining for alkaline phosphatase is carried out through an enzymatic reaction with Bromo-4-Chloro-3′-Indolylphosphate p-Toluidine Salt (BCIP) and Nitro-Blue Tetrazolium Chloride (NBT). BCIP is hydrolyzed by alkaline phosphatase to form an intermediate that undergoes dimerization to produce an indigo dye. The NBT is reduced to the NBT-formazan by the two reducing equivalents generated by the dimerization. Together these reactions produce an intense, insoluble black-purple precipitate when reacted with alkaline phosphatase.

FIG. 16-C shows a representative image of passage 2 cells isolated and expanded from placental composition derived from the chorionic membrane staining positively for alkaline phosphatase after osteoinduction.

### Example 23

### Viable Cell Content in Multiple Lots of Minced Placental Composition

The quantity of viable cells in 29 lots of placental composition processed as in Example 1 and Example 4 were measured. Placental composition produced as in Example 4 contains the placental cells within the small tissue pieces generated by mincing. In order to quantify these placental cells using a hemocytometer and trypan blue exclusion, the cells must first be released from the tissue pieces by collagenase digestion.

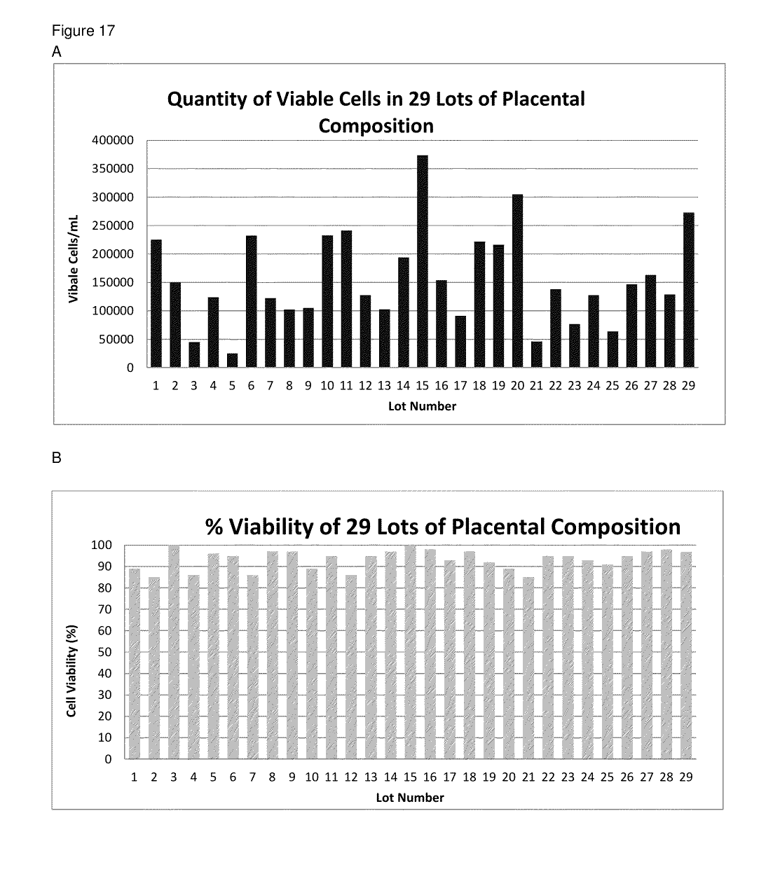

A vial of each lot was thawed for and digested with 250 U/mL collagenase type II (Worthington) in DMEM for 40 min at 37° C. with shaking. The digestion suspensions were then filtered through 100 μm cell strainers, and the strainers were washed with DMEM. The filtrates were centrifuged at 411 rcf for 8-10 min. The supernatants were removed, and the pellets were re-suspended in DMEM. The supernatants were counted using a hemocytometer by trypan blue exclusion (results shown in FIG. 17).

Results in FIG. 17 show a high quantity of placental cells and high % viability in the placental composition processed by mincing alone with no collagenase digestion of the chorionic membrane.

### Example 24

### Inhibition of Pro-Inflammatory TNF-α by Minced and Digested Placental Composition

Placental composition was tested for response to inflammation to mimic the inflammatory conditions found in chronic wounds. The inhibition of TNF-α production by PBMCs (peripheral blood mononuclear cells) was measured to determine the immunmodulatory capacity of the placental composition.

Two placentas processed as in Example 1 were each split in half before antibiotic treatment. One half of each was processed into placental composition as in Example 3 (digested placental composition) and half was processed into placental composition as in Example 4 (minced placental composition). The minced and digested placental compositions with their different configurations were compared for their respective ability to modulate inflammation.

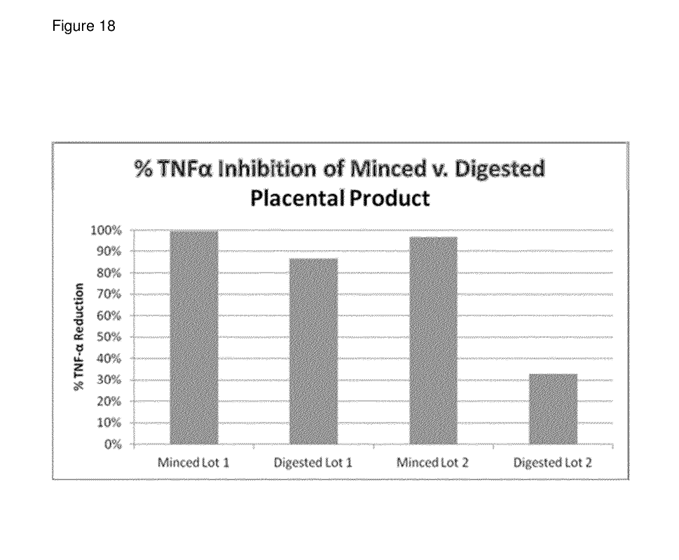

CD3 and CD28 were added to PBMCs to stimulate the production of inflammatory cytokines such as TNF-α. The PBMCs were then cultured with either minced or digested placental composition. As a positive control, stimulated PBMCs were cultured in DMEM. As a negative control, PBMCs without stimulatory CD3 and CD28 were cultured in DMEM. The cultures were incubated for 60-84 hr until the positive control culture displayed PBMC aggregation. The culture samples were collected and centrifuged at 16,000 rcf for 10 min. The supernatants were collected and tested for TNF-α content by ELISA (results shown in FIG. 18).

Results as shown in FIG. 18 show that both lots of minced and digested placental composition significantly inhibited TNF-α production in relation to the positive control. Minced placental composition more robustly inhibited the inflammatory cytokine than digested placental product potentially due to the fact that the placental cells in minced placental composition remain embedded in their native tissue unlike most of the placental cells in digested placental composition.

### Example 25

### Elastase Inhibition by Minced Placental Composition

Chronic wounds often have disproportionately high levels of proteolytic enzymes such as MMPs (matrix metalloproteinases) and elastase that prevent the wound from progressing into the regenerative phase. Minced placental product, processed by Example 1 and Example 4, was tested in an elastase inhibition assay for its ability to mediate protease activity.

A vial of minced placental composition was thawed and combined with human neutrophil elastase in HEPES buffer to a final concentration of 0.01 mM and elastase substrate to a final concentration of 5.7 mM (N-Methoxysuccinyl-Ala-Ala-Pro-Val-P-Nitroanilide, Sigma # M4765). For a positive control, cryoprotectant (5% DMSO, 5% HSA in saline) was used in lieu of placental composition. The samples were incubated at 37° C. incubator with shaking overnight.

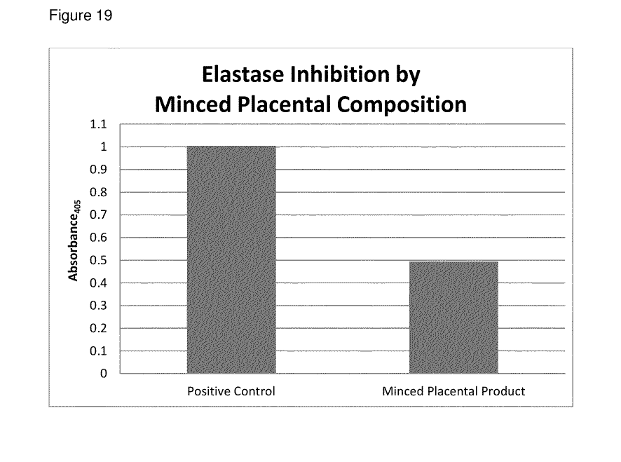

Substrate degradation was measured by measuring OD at 405 nm. The results are shown in FIG. 19. Enzymatic substrate hydrolysis results in an increase in absorbance. Enzymatic substrate hydrolysis was lowered in minced placental composition as compared to the positive control confirming the ability of minced placental composition to regulate the chronic wound environment through inhibition of proteases.

### Example 26

### VEGF Content of Minced and Digested Placental Compositions

The quantity of the angiogenic growth factor VEGF in minced and digested placental compositions was determined for two lots.

Two placentas processed as in Example 1 were each split in half before antibiotic treatment. One half of each was processed into placental composition as in Example 3 (digested placental composition) and half was processed into placental composition as in Example 4 (minced placental composition).

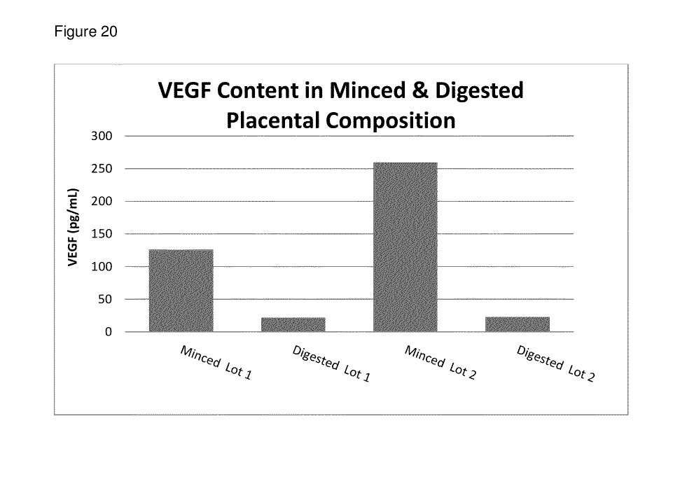

Minced and digested placental compositions were thawed and centrifuged at 16,000 rcf for 10 min. The supernatants were tested for VEGF expression using a VEGF ELISA kit (shown in FIG. 20).

The results are shown in FIG. 20 and indicate that both minced and digested placental compositions contain VEGF, though minced composition contains a greater quantity for both tested lots. Because minced placental composition is not partially digested in collagenase as placental composition prepared as in Example 2 or Example 3, minced placental composition likely retains a greater quantity of native growth factors and extracellular matrix proteins.

### Example 27

### VEGF Content of Minced and Digested Placental Composition after Lysis in Guanidine HCl

In order to access the VEGF of minced and digested placental compositions that is embedded in the minced tissue pieces, placental compositions were subjected to lysis in guanidine HCl.

Two placentas processed as in Example 1 were each split in half before antibiotic treatment. One half of each was processed into placental composition as in Example 3 (digested placental composition) and half was processed into placental composition as in Example 4 (minced placental composition).

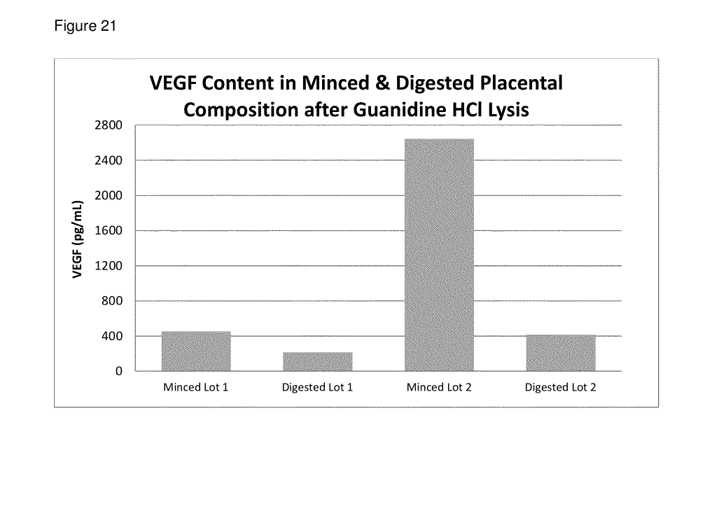

A protease inhibitor tablet (Complete protease inhibitor cocktail tablets, Roche #04693124001) was added to 10 mL of chilled 8 M Guanidine HCl (hereafter referred to as GuHCl). Minced and digested placental compositions were thawed and centrifuged at 16,000 rcf for 12 min. Supernatants were removed and moved into fresh tubes and placed on ice. The tubes containing the cell/tissue pellets were snap frozen by placing in liquid nitrogen for 5 min. A chilled lysis bead and GuHCl to a final concentration of 4M was added to each pellet. The pellets were placed in the chilled chamber of the Tissue Lyser, and the samples were lysed for 6 min at 50 Hz. The corresponding chilled supernatants were added back to the lysed pellets and incubated at 4° C. overnight with rotation. Exchange buffer was prepared by adding a protease inhibitor tablet to 10 mL of chilled D-PBS. The lysed placental compositions were centrifuged at 16,000 rcf at 4° C. in a microcentrifuge for 12 min, and the supernatants were collected. A desalting column (Zeba desalting columns, Thermo Scientific #89892) was prepared for each sample per manufacturer's instructions using exchange buffer to wash the storage buffer off the columns. Each supernatant was applied to a column and centrifuged at 913 rcf for 12 min. The VEGF concentration was measured in the desalted supernatants by ELISA (show in FIG. 21).

Results in FIG. 21 show VEGF content higher than is usually tested when the samples are not subjected to GuHCl lysis indicating that there is VEGF left embedded in the tissue pieces of the placental compositions. As in the results displayed in FIG. 20, minced placental composition contained more VEGF than digested placental composition for both tested lots.

### Example 28

### bFGF Content of Minced Placental Composition after Lysis in Tissue Extraction Buffer

In order to access the bFGF of minced placental compositions that is embedded in the minced tissue pieces, placental compositions were subjected to lysis in tissue extraction buffer. Previous experiments showed the bFGF ELISA to be incompatible with guanidine HCl treatment so alternative lysis buffers were explored.

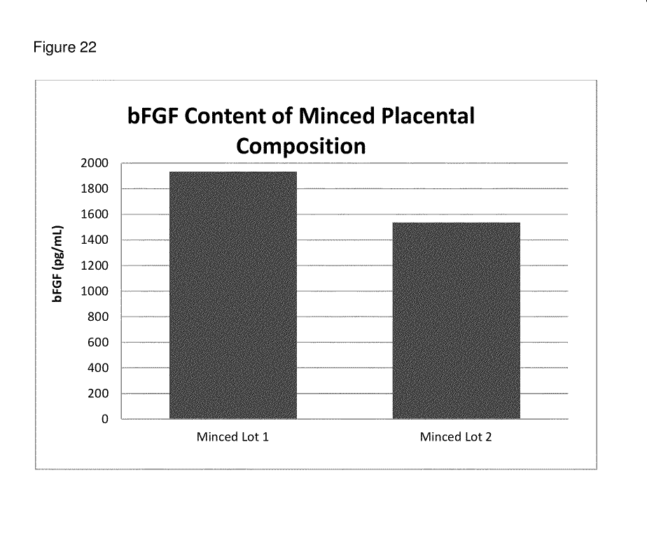

An extraction solution was prepared by adding 1 protease inhibitor tablet to 10 mL chilled tissue extraction buffer (Thermo Scientific #78510). Minced placental composition, prepared as in Example 1 and Example 4, were thawed and centrifuged at 16,000 rcf for 12 min. The supernatants were moved to new tubes and kept on ice. The cell/tissue pellets were snap frozen along in liquid nitrogen for 5 min. An amount of extraction buffer equivalent to the amount of placental composition and a chilled lysis bead were added to each tube. Tubes were placed in a chilled tissue lyser chamber and the samples were lysed for 6 minutes at 50 Hz. To the corresponding lysed tissue, the supernatants were added and were centrifuged at 16,000 rcf for 12 min. The supernatants were collected and tested for bFGF with DuoSet ELISA kit (R&D Systems, Minneapolis Minn.). Results are found in FIG. 22.

High levels of bFGF were found in both lots of minced placental compositions.

### Example 29

### Sustained Growth Factor Release from Minced and Digested Placental Compositions

To demonstrate the ability of minced and digested placental compositions to sustain growth factor release over time, the compositions were cultured for two weeks and VEGF content was measured.

Two placentas processed as in Example 1 were each split in half before antibiotic treatment. One half of each was processed into placental composition as in Example 3 (digested placental composition) and half was processed into placental composition as in Example 4 (minced placental composition).

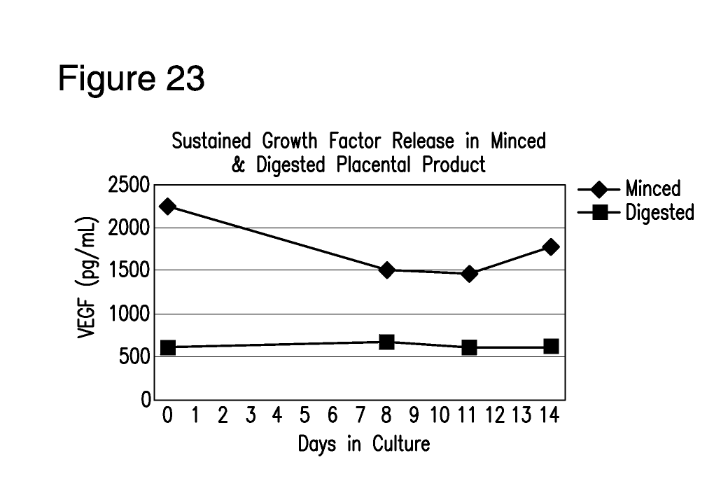

Minced and digested placental compositions were thawed and added to 6-well culture plates with DMEM, 1% FBS. The plates were placed in a 37° C., 5% CO₂ incubator. After 0 days, 8 days, 11 days, and 14 days, the plates were removed from incubation and samples were collected. The collected samples were centrifuged at 16,000 rcf for 12 min in a microcentrifuge. Supernatants were transferred to fresh tubes and both the pellets and supernatants of each sample were stored at −80° C. until further processing. Each sample was processed by guanidine HCl treatment as described in Example 28. VEGF concentration was measured in desalted samples by ELISA and the results are depicted in FIG. 23.

Both minced and digested placental compositions displayed sustained release of VEGF over a period of two weeks. Minced placental composition released a greater quantity of VEGF than digested placental composition.

### Example 30

### Cell Proliferation of Minced Placental Composition

The cell proliferation capabilities of minced placental compositions were tested. Placental cells were seeded and cultured for 14 days.

Vials of placental composition, prepared as in Example 1 and Example 4, were thawed and digested for 20 min with rocking at 37° C. Each sample was digested either in 250 U/mL Serva type II collagenase or 250 U/mL Worthington type II collagenase. The digested compositions were poured over 100 μm cell strainers, and the strainers were washed with DMEM. The digested compositions were centrifuged for 10±min at 411 rcf. The supernatants were removed and re-suspended in DMEM. Each cell suspension was counted with a hematocytometer by trypan blue exclusion.

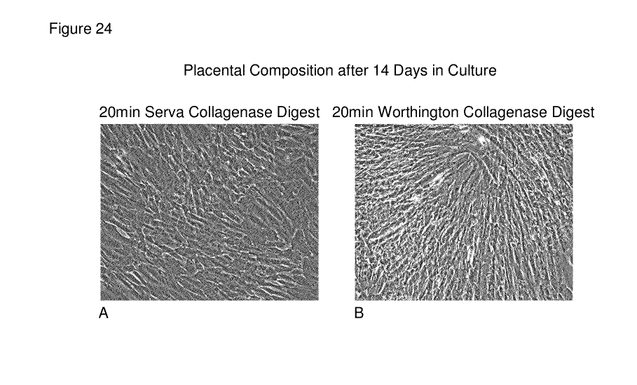

Each group of processed cells was seeded in T25 flasks and cultured for 14 days in an incubator. Results can be seen in FIG. 24 A-B. Cells derived from the minced placental composition had established cell colonies after 14 days in cell culture.

### Example 31

### Carrier Attachment of Minced Placental Composition

The compatibility of different carriers for use with minced placental compositions in bone regeneration was tested.

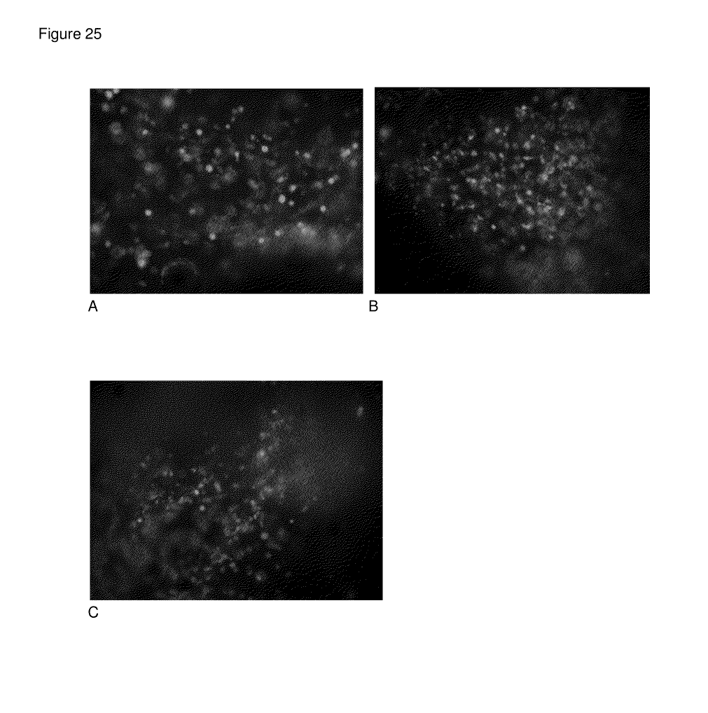

Carrier materials were placed in individual wells of a 24-well Nunclon multidish. The carrier materials included HA-TCP-Collagen Foam (1 cm×1 cm piece) and TranZgraft cancellous granules. Minced placental composition, produced as in Example 1 and Example 4, was thawed and added to each of to the different materials. D-PBS was added to the samples and the samples were left at room temperature for one hour to mimic the maximum amount of time surgeons are recommended to leave placental composition mixed with carrier after thawing. The liquid was removed from the wells. The materials were stained for viable and non-viable cells using the LIVE/DEAD® assay (Life Technologies, Grand Island, N.Y.). 10 μL of the ethidium homodimer-1 solution (Component B, 2 mM EthD-1 stock solution) and 5 μL of the calcein AM solution (Component A, 4 mM calcein AM stock solution) was added to 10 mL D-PBS. This staining solution was added to the samples until samples were completely covered. Samples were protected from the light and left at room temperature for 30 min. The staining solution was removed from the wells and washed with D-PBS, and the samples were viewed under a fluorescence microscope. Live cells fluoresced green and dead cells fluoresced red. FIGS. 25 A and B shows the placental composition on HA-TCP-Collagen Foam, and FIG. 25 C shows the placental composition on TranZgraft. Pictures of live and dead cells were taken in the same location each time.

Results show that minced placental composition is highly compatible with a variety of commonly used osteoconductive scaffolds and remains viable in combination with these carriers after 1 hour.

### Example 32

### Stability Testing of Final Chorionic Placental Composition

Stability of the final composition was tested to evaluate possible product degradation during long term storage of 24 months at −80° C. Indicators of product degradation are reduced numbers of viable cell and decreased cell viability. The number of viable cells and cell viability were determined after completion of the initial freeze down and 24 months post-freezing for three lots of placental composition. In addition, sterility testing was performed 12 months and 24 months post-freezing.

Three placentas were processed separately according to the procedures described in Example 1 and 2, then packaged in borosilicate glass vials, and frozen in 5% DMSO in saline at −80° C. Placental composition of each of the three lots was thawed directly after completion of the initial freeze down and 24 months post-freezing, and counted for viable and dead cells using a hemocytometer and trypan blue staining. Cell viability was calculated for each sample. Results in Table 7 show the number of viable cells and the cell viability of final compositions after completion of the initial freeze-down and 24 months post-freezing.

The final composition showed minimal to no degradation after 24 months of storage at −80° C. Two years after initial cryopreservation, every lot of tested final composition still contained more than 100,000 viable cells per mL and had cell viability of at least 70%.

TABLE 7
Stability of Final Composition after Long-Term Storage

| | Initial Testing | | Testing At 24 Months | |
|---|---|---|---|---|
| Lot | Viable | Cell Viability | Viable | Cell Viability |
| Number | cells/mL | [%] | cells/mL | [%] |
| Lot 1 | 187,000 | 70.3 | 133,000 | 81.6 |
| Lot 2 | 274,000 | 76.6 | 261,000 | 84.6 |
| Lot 3 | 221,000 | 74.3 | 259,000 | 75.8 |
| Average * | 227,000 | 73.7 | 218,000 | 80.7 |

* Average cell numbers were rounded

Samples of each lot were also tested for sterility at two different time points post-freezing (12 months and 24 months). Testing was performed by an approved test facility. All samples tested negative (Table 8).

TABLE 8
Sterility Results for Final Product at Different Time Points Post-Freezing

| Lot Number | 12 Months | 24 Months |
|---|---|---|
| Lot 1 | Negative | Negative |
| Lot 2 | Negative | Negative |
| Lot 3 | Negative | Negative |

### Example 33

### Tissue Piece Sizes of Minced Placental Composition

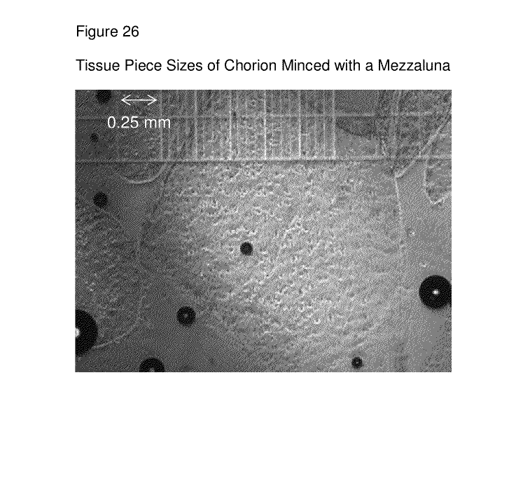

Tissue piece size of a placental composition prepared as in Example 1 and Example 4 (minced with herb mincer) was compared to a placental composition prepared as in Example 1 and Example 5 (minced with mezzaluna). Final composition was thawed, and 10 μL were transferred to a hematocytometer. 5 μL trypan blue was added, and a circular coverglass was placed on top of the cell suspension. The size of tissue pieces for final composition prepared using a herb mincer or mezzaluna were compared. The average size for tissue pieces of chorion minced with the herb mincer was 0.421 mm² (n=14) and for chorion minced with a mezzaluna 1.137 mm² (n=14). Tissue pieces of chorion minced with the mezzaluna were significantly larger than tissue pieces of chorion minced by the herb mincer (see FIG. 26).

### Example 34

### Use of Placental Tissue Composition in Spinal Fusion Surgery

Background.

A 34-year old female presented with constant lower back pain, buttock pain, and paresthesia into her right lower extremity posteriorly extending to her 2 lateral toes that had remained unresolved for 7.5 months after injury.

Evaluation.

The physical exam revealed 2+ patellar reflexes bilaterally, with 1+Achilles reflexes bilaterally, diminished right foot dorsiflexion and positive straight leg raise on the right. X-rays and a CT were obtained which revealed right foraminal narrowing and collapse and no evidence of spondylosis.

Diagnosis.

L5-51 herniation with right foraminal narrowing and collapse. Right L5 and 51 radiculopathy with mechanical instability of L5-51. Right ankle weakness concordant to disc herniation and right lower extremity radiculomyelopathy.

Surgical Procedure.

Approximately 12 months post-injury, the right sided interpedicular space at L5-S1 was microsurgically dissected and the disc material was removed via facetectomy, foraminotomy and microdiscetomy. Preparation of the bone graft was completed which consisted of 15 cc of demineralized bone matrix (DBM) mixed with 1 mL of placental composition. Both grafting materials are radiolucent at implantation. Once decompression was completed, the endplates were decorticated and an oblique 8 mm T-PAL PEEK spacer was packed with 5 cc of DBM+placental composition and placed transforaminally. Extensive decortication along the left dorsolateral mass of L5-S1 was completed through a microsurgical dissection. The remaining 10 cc of DBM+placental composition was applied to the left dorsolateral mass for onlay fusion. Rod and cap assembly was completed.

Follow-Up.

Coronal and sagittal CTs at 6 months post-surgery show evidence of early fusion through the left facet joint and early interbody trabecular bone formation. The interbody bone graft and hardware are in optimal position. CT thin sections at an early time point can identify non-union but is not as sensitive at identifying fusion. Early bone fusion can typically be visualized on CT in as little as 6-12 months minimally (Williams A, Gornet M, Burkus K. CT Evaluation of Lumbar Interbody Fusion: Current Concepts. AJNR Am J Neuroradiol 2005; 26:2057-2066).

### Example 35

### Compositions for Use in Treating Tunnel Wounds

A patient with a tunnel wound is treated by administration once a week with 1 mL of the cryopreserved placental composition for at least 4 to 6 weeks. Treatment is stopped when there is re-epithelialization and closure of the wound.

### Example 36

### LPS-Induced TNF-α Secretion by Placental Membrane Cells

As described herein, fetal macrophages present in the amnion and chorion are a major source of tissue immunogenicity. Without being bound by theory, the present inventors believe that removal of CD14+ fetal macrophages from placental membrane and compositions prevents activation of lymphocytes and decreases the level of inflammatory cytokine secretion and tissue immunogenicity. Macrophages in fetal placental membranes respond to bacterial LPS by secretion of inflammatory cytokines such as TNF-α. Therefore, secretion of TNF-α in response to LPS is used here to characterize tissue immunogenicity of placental membranes at each critical manufacturing step. Samples from each manufacturing step included trophoblast (T), amnion with chorio trophoblast (ACT), choriotrophoblast (CT), chorion (CM), and amnion (AM).

Pieces of placental membranes (2 cm×2 cm) representing intermediates and final products were placed in tissue culture medium and exposed to bacterial LPS (1 μg/mL) for 20-24 hr. Tissue culture supernatants were then collected and tested for the presence of TNF-α using a TNF-α ELISA kit (R&D Systems) according to the manufacturer's protocol. Human hPBMCs (SeraCare) known to contain monocytes responding to LPS by secretion of high levels of TNF-α were used as a positive control. hPBMCs and placental tissues without LPS were also included as controls in the analysis. In this assay, TNF-α detected in the culture medium from greater than 70 pg/cm² (corresponding to 280 pg/mL) for both spontaneous and LPS-induced TNF-α secretion was considered immunogenic (Fortunato, et al. 1996).

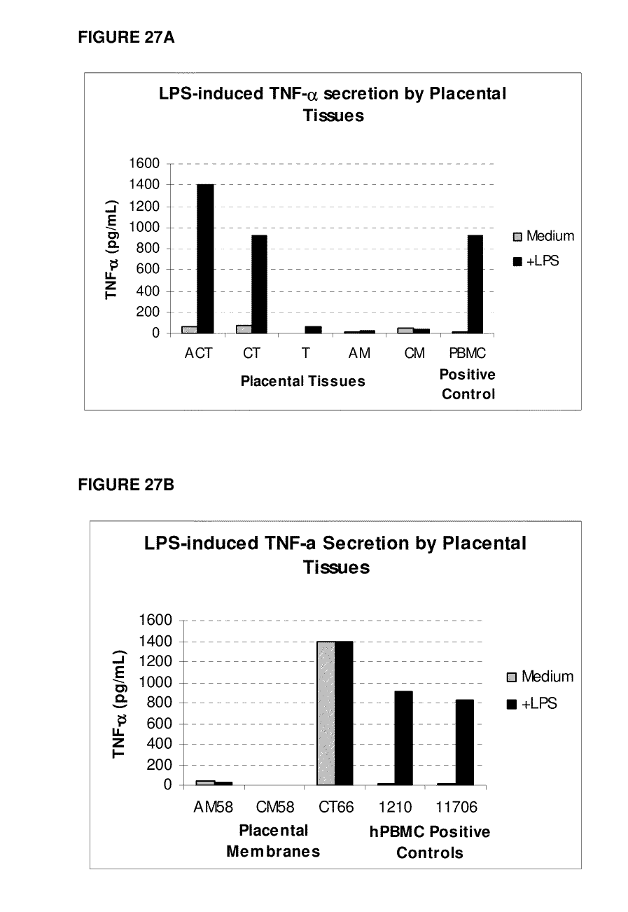

As depicted in FIGS. 27A and 27B, the manufacturing process serially reduces immunogenicity of the placental product. AM and CM had only 23.5 and 40 pg/ml TNF-α secretion as compared to ACT and CT at 1397.1 and 917.2 pg/ml, respectively. Tissues cultured in medium without LPS show the basal level of TNF-α secretion. PBMCs, which are known to secrete high levels of TNF, were used as a positive control.

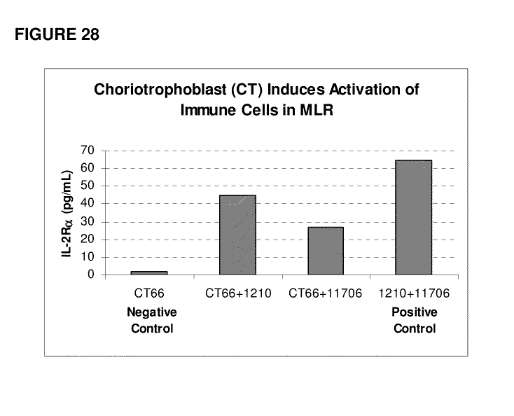

Choriotrophoblast membranes (CT) include the chorion membrane with an intact trophoblast layer. CT membrane which secreted high levels of TNF-α, was tested in MLR against two different PBMC donors (FIG. 28). CT cells were cultured with PBMCs for 4 days. IL-2Rα was measured in cell lysates as a marker of T-cell activation. Positive control: a mixture of PBMCs derived from 2 different donors. Results of this assay, as seen in FIG. 28, showed a correlation with the MLR data: tissues that produce high levels of TNF-α in response to LPS are immunogenic in the MLR assay.

In conclusion, the low levels of TNF-α and the absence of the response to LPS by AM and CM indicates the exemplary cryopreservation method described in the current technology eliminates viable functional macrophages from the amniotic and chorionic membranes, which ensures the safety of such an allogeneic product.

1-110. (canceled)

111. A cryopreserved placental tissue composition comprising:
disrupted placental tissue comprising
(i) one or more placental cells,
(ii) one or more therapeutic factors,
(iii) one or more extracellular matrix components; and
(iv) tissue pieces comprising (i), (ii), (iii) or combinations thereof;
wherein, after subsequent thawing of the cryopreserved placental tissue composition, greater than 40% of the placental cells are viable and the composition is depleted in functional immunogenic cells.

112. The cryopreserved placental tissue composition of claim 111 further comprising a cryopreservation agent.

113. The cryopreserved placental tissue composition of claim 1, wherein the placental tissue is chorionic or amnionic tissue.

114. The composition of claim 1, wherein the composition is applied to a subject in need of a therapeutic benefit, and wherein the one or more placental cells, therapeutic factors, extracellular matrix components or combinations thereof are present in an amount effective to:
(i) a reduction in the amount and/or activity of pro-inflammatory cytokines;
(ii) an increase in the amount and/or activity of anti-inflammatory cytokines;
(iii) a reduction in the amount and/or activity of reactive oxygen species;
(iv) an increase in the amount and/or activity of anti-oxidant agents;
(v) a reduction in the amount and/or activity of proteases;
(vi) an increase in cell proliferation;
(vii) an increase in angiogenesis; and/or
(viii) an increase in cell migration.

115. The cryopreserved placental tissue composition of claim 111, wherein the composition is used in a surgical procedure selected from the group consisting of a tissue graft procedure, tendon surgery, ligament surgery, bone surgery, neurosurgery, and spinal surgery.

116. The cryopreserved placental tissue composition of claim 111, wherein the cryopreserved placental composition can be thawed at room temperature in less than 10 minutes.

117. The cryopreserved placental tissue composition of claim 116, wherein the cryopreserved placental composition can be thawed at room temperature in less than one minutes.

118. The cryopreserved placental tissue composition of claim 38, wherein the one or more tissue pieces are about 0.01 mm² to about 5 mm² in size.

119. The cryopreserved placental tissue composition of claim 111, wherein the viability of the cells is maintained for at least 24 months when stored frozen.

120. The cryopreserved placental tissue composition of claim 111, wherein the extended period of time is about 6 to about 36 months.

121. The cryopreserved placental tissue of claim 111, wherein the viability of the cells is substantially maintained upon thawing.

122. A method of treating a tunneling wound on a subject in need thereof comprising administering to the site of the tunnel wound the cryopreserved placental composition of claim 111.

123. A method of treating a wound or tissue defect of a subject in need thereof comprising administering the cryopreserved placental composition of claim 111.

124. The method of claim 123, wherein the wound is selected from the group consisting of: lacerations, scrapes, burns, incisions, punctures, wound caused by a projectile, an epidermal wound, skin wound, chronic wound, acute wound, external wound, internal wound, congenital wound, ulcer, pressure ulcer, diabetic ulcer, tunnel wound, wound caused during or as an adjunct to a surgical procedure, venous skin ulcer, spinal injury, ocular wound, and avascular necrosis.

125. A kit for treating a wound or a tissue defect comprising:
a) a placental tissue composition according to claim 111 in a pharmaceutically acceptable container; and
b) instructions for administering the placental tissue composition for treating the wound or the tissue defect.

126. A process for preparing a placental tissue composition comprising the steps of:
a. providing a placental tissue comprising placental cells, therapeutic factors, and extracellular matrix components;
b. disrupting at least a portion of the placental tissue to form a placental dispersion comprising placental tissue pieces, placental cells, therapeutic factors and extracellular matrix components; and
c. cryopreserving the placental dispersion to form the cryopreserved placental tissue composition of claim 111.

127. The process of claim 126, further comprising the steps of, prior to the disrupting step,
a1. digesting the placental tissue with at least one enzyme to form a suspension of placental cells and placental tissue pieces; and
a2. separating the placental cells and the placental tissue pieces, and
wherein in step (b) the disrupting step comprises disrupting the placental tissue pieces separated in step (a2), and then after (b) but before (c) combining the placental cells separated in (a2) and disrupted tissue pieces to form the placental dispersion.

128. The process of claim 126, wherein the step of cryopreserving comprises adding a cryopreservation agent.

129. The process of claim 126, wherein steps further comprise adding serum albumin to the placental composition.

130. The process of claim 128, wherein the placental tissue is disrupted by mincing.
